(19) United States
(12) Patent Application Publication
(10) Pub. No.: US 2026/0263553 A1
(43) Pub. Date: Sep. 10, 2026

(54) METHODS OF TREATING A LOCALIZED INFECTION WITH LOCALLY ADMINISTERED ANTIBIOTICS

(71) Applicant: Osteal Therapeutics, Inc., Dallas, TX (US)

(72) Inventors: Brian C. DE BEAUBIEN, Fenton, MI (US); Kevin WARNER, Dallas, TX (US); Jude PAGANELLI, Coronado, CA (US); William C. PUTNAM, Frisco, TX (US); Daniel PFLASTER, Charlotte, VT (US)

(73) Assignee: Osteal Therapeutics, Inc., Dallas, TX (US)

(21) Appl. No.: 19/157,972

(22) PCT Filed: Feb. 21, 2024
(86) PCT No.: PCT/US24/16710

§ 371 Date: Aug. 19, 2025

Related U.S. Application Data
(60) Provisional application No. 63/486,186, filed on Feb. 21, 2023.
(60) Provisional application No. 63/493,577, filed on Mar. 31, 2023.
(60) Provisional application No. 63/519,923, filed on Aug. 16, 2023.
(60) Provisional application No. 63/596,130, filed on Nov. 3, 2023.
(60) Provisional application No. 63/613,533, filed on Dec. 21, 2023.

(51) Int. Cl. A61K 38/14; A61K 9/08; A61K 31/7036; A61P 31/04

(52) U.S. Cl. CPC A61K 38/14; A61K 9/08; A61K 31/7036; A61P 31/04

(57) ABSTRACT

A method of treating a local infection in a biological tissue of a human patient in need thereof is described. The method may include locally administering to the human patient a daily dose of at least one antibiotic that substantially exceeds a maximum recommended daily dose of the at least one antibiotic and providing a peak or trough serum concentration less than or equal to a maximum safe level for serum concentration of the at least one antibiotic.

### CROSS-REFERENCE TO RELATED APPLICATIONS

This application claims priority to U.S. Provisional Application No. 63/486,186, filed Feb. 21, 2023, U.S. Provisional Application No. 63/493,577, filed Mar. 31, 2023, U.S. Provisional Application No. 63/519,923, filed Aug. 16, 2023, U.S. Provisional Application No. 63/596,130, filed Nov. 3, 2023, and U.S. Provisional Application No. 63/613,533, filed Dec. 21, 2023, the contents of each of which are incorporated herein by reference in their entirety.

### TECHNICAL FIELD

The present disclosure generally relates to methods, doses, and concentrations of antibiotic irrigation of biological tissue. In particular, the description relates to methods, doses, and concentrations of irrigation of localized antibiotics in and around wounds, including surgical wounds, traumatic wounds, wounds caused by or worsened by infection and wounds containing infected tissue or medical devices.

### BACKGROUND

Localized infection, including septic arthritis, periprosthetic joint infection (PJI), breast implant infection (BII), and fracture related infection (FRI), is a rare but devastating pathology associated with severe complications. Successful management of localized infection includes surgical intervention and systemic antibiotics in most cases. An important part of the surgical procedure is reduction of the bacterial bioburden. Bioburden removal is accomplished through intraoperative irrigation and surgical debridement of infected tissue. Antibiotic therapy for the treatment of the infection is commonly systemic antibiotics (administered orally or intravenously). Localized antibiotics may also be applied during the surgical procedure, but localized therapeutic concentrations are difficult to maintain post-operatively. It is the systemic antibiotic therapy that is considered the treatment of the infection.

An infected implant may be replaced or retained at the time of treatment depending on the clinical presentation. Many pathogens commonly found in PJI, BII, FRI, or other infections involving implants are known to form a protective structure called a biofilm in the presence of an implanted medical device. Such biofilms are not susceptible to antibiotics administered systemically; therefore, the infected implant must be surgically removed as part of any treatment if the infection is to be fully eradicated with a high degree of success. In cases where the infection is well established, implants are typically replaced in a one-stage or two-stage exchange procedure. In acute cases, cases of infection in medically compromised patients and cases of implants which are unusually difficult to remove, the most common surgical treatments retain the implants and are known as irrigation and debridement (I&D) or debridement, antibiotics and implant retention (DAIR). Replacing the implant is more invasive than retaining the implant, but it reduces bacterial bioburden, particularly biofilm, and improves outcomes compared to implant retention.

Infections of traumatic wounds are a common occurrence, as are surgical site infections (SSI) which occur post-operatively following virtually all types of surgical procedures. Such infections may be superficial and relatively easy to treat successfully, while like PJI, others involve deep tissue with or without surgically implanted devices and become very difficult to eradicate.

In systemic antibiotic therapy for musculoskeletal infection, the administered dose in a 24-hour period (e.g., total daily dose) and concentration are strictly limited by the maximum safe concentrations of the antibiotics in the circulatory system, and the resulting dose and concentration at the site of local infection is further limited by pharmacokinetics.

In view of the foregoing, there is a need for improved methods, doses, and concentrations of antibiotic irrigation of wounds.

### SUMMARY

In accordance with some implementations of the disclosed subject matter, therapeutic methods, doses and concentrations of antibiotic irrigation are provided for treatment of various wounds.

The present disclosure is directed to a method of treating a localized infection of a human patient. In an aspect, the method includes locally administering a solution to the human patient in a 24-hour period a dose of at least one antimicrobial agent that substantially exceeds a maximum recommended daily systemic dose of the at least one antimicrobial agent, and during the 24-hour period providing a peak or trough serum concentration less than or equal to a maximum safe level for serum concentration of the at least one antimicrobial agent.

Another aspect of the present disclosure is a method of treating a localized infection of a human patient by locally administering to the human patient a locally administered concentration of at least one antimicrobial agent that exceeds a maximum recommended concentration of the at least one antimicrobial agent and providing a peak or trough serum concentration less than a maximum safe serum concentration of the at least one antimicrobial agent.

The present disclosure is further directed to a method of treating a musculoskeletal infection of a human patient by administering to the human patient at least one antimicrobial agent at a locally administered concentration that exceeds a minimum biofilm eradication concentration (MBEC) of the at least one antimicrobial agent and providing a peak or trough serum concentration of the at least one antimicrobial agent less than a maximum safe serum concentration for the at least one antimicrobial agent.

Another aspect of the present disclosure is a method of treating a localized infection of a human patient by locally administering to the human patient, in a 24-hour period, a total dose of a first antibiotic that substantially exceeds a maximum recommended systemic daily dose of the first antibiotic and providing a peak or trough serum concentration during the 24-hour period less than or equal to a maximum safe serum concentration of the first antibiotic.

The present disclosure additionally provides a method of treating a localized infection of a human patient in need thereof by locally administering to the human patient, in a 24-hour period, a total dose of a first antibiotic that substantially exceeds a maximum recommended daily systemic dose of the first antibiotic and a total dose of a second antibiotic that substantially exceeds a maximum recommended daily dose of the second antibiotic and providing a peak or trough serum concentration, during the 24-hour period, less than or equal to a maximum recommended safe serum concentration of the first antibiotic and the second antibiotic.

A further aspect of the present disclosure is a method of treating a localized infection of a human patient by locally administering to the human patient, in a 24-hour period, a total dose of at least two antibiotics that substantially exceeds a maximum recommended daily systemic dose of each of the at least two antibiotics; and providing a peak or trough serum concentration, during the 24-hour period, less than or equal to a maximum safe serum concentration of each of the at least two antibiotics.

An additional aspect of the present disclosure is a method of treating a localized infection of a human patient by locally administering to the human patient, in a 24-hour period, a total dose of a first antibiotic that substantially exceeds a maximum recommended systemic daily dose of the first antibiotic; and providing a peak or trough serum concentration during the 24-hour period less than or equal to a maximum safe serum concentration of the first antibiotic.

An even further aspect of the present disclosure is a method of treating a localized infection of a human patient in need thereof by locally administering to the human patient, in a 24-hour period, a total dose of a first antibiotic that substantially exceeds a maximum recommended daily systemic dose of the first antibiotic and a total dose of a second antibiotic that substantially exceeds a maximum recommended daily dose of the second antibiotic and providing a peak or trough serum concentration, during the 24-hour period, less than or equal to a maximum recommended safe serum concentration of the first antibiotic and the second antibiotic.

The present disclosure additionally provides a method of treating a localized infection of a human patient by locally administering to the human patient, in a 24-hour period, a total dose of at least two antibiotics that substantially exceeds a maximum recommended daily systemic dose of each of the at least two antibiotics; and providing a peak or trough serum concentration, during the 24-hour period, less than or equal to a maximum safe serum concentration of each of the at least two antibiotics.

The present disclosure further provides a method of treating a biological tissue of a human patient by locally administering to the human patient, in a 24-hour period, a total dose of at least one therapeutic agent that substantially exceeds a maximum recommended systemic daily dose of the at least one therapeutic agent and providing a peak or trough serum concentration less than or equal to a maximum safe serum concentration of the at least one therapeutic agent.

The present disclosure also provides a method of treating a localized infection in a biological tissue of a human patient by locally administering to the human patient, in a 24-hour period, a total dose of at least one antibiotic that exceeds 3,000 mg in the 24-hour period.

An aspect of the present disclosure includes a method of treating a localized infection in a human patient by locally administering to the human patient, in a 24-hour period, a total dose of vancomycin that exceeds 3,000 mg/day and/or a total dose of tobramycin that exceeds 100 mg in the 24-hour period.

Another aspect of the present disclosure includes a kit for treating a localized infection in a human patient. The kit may include at least one antibiotic, a treatment delivery system and/or an irrigation device configured to locally irrigate the localized infection with at least one dose of the at least one antibiotic, and instructions for administering the at least one dose of the at least one antibiotic.

An additional aspect of the present disclosure includes a method of treating a localized infection in a human patient by locally administering to the human patient two or more doses within a 24-hour period of at least one antibiotic at a concentration that exceeds a MBEC level for the at least one antibiotic for the local infection.

A further aspect of the present disclosure includes a method of treating a localized infection of a human patient by locally administering to the human patient two or more doses of at least one antibiotic within 24 hours, where an AUC/MBEC is maintained at the site of the localized infection for at least 5 hours a day.

A further aspect of the present disclosure includes a method of treating a localized infection in a human patient by locally administering to the human patient for a period of two or more days, at least one antibiotic at a concentration that exceeds a MBEC for the at least one antibiotic for the local infection for at least a first day, and the at least one antibiotic at a concentration less than the concentration of the at least one antibiotic on the first day. The concentration of the at least one antibiotic on subsequent days may exceed a minimum inhibitory concentration of the at least one antibiotic for the local infection.

The present disclosure provides a method of treating a local infection of a human patient in need thereof by administering by local irrigation to the human patient at least a 25 mg dose of vancomycin at a concentration of 3,000 mcg/mL or greater and providing less than a 30 mcg/mL trough serum concentration in the patient for 24 hours following administration.

The present disclosure further provides a method of treating a local infection of a human patient in need thereof by administering by local irrigation to the human patient a dosage of vancomycin and tobramycin. The dosage may include at least a 50 mg dose of vancomycin at a concentration of 3,000 mcg/mL or greater and at least a 50 mg dose of tobramycin at a concentration of 2,000 mcg/mL or greater. The administering provides a trough serum concentration less than 30 mcg/mL of vancomycin and a peak serum concentration less than 10 mcg/mL of tobramycin in the patient for 24 hours following administration.

A further aspect of the present disclosure includes a method of treating a localized infection in a human patient by identifying at least one microbe within the localized infection; and locally administering to the human patient two or more antibiotics, where at least one of the antibiotics is administered at a concentration that exceeds a MBEC level for the at least one antibiotic for the at least one microbe.

The present disclosure further provides a method of treating a localized infection in a human patient by locally administering to the human patient two or more antibiotics, where at least one of the antibiotics is administered at a concentration that exceeds a MBEC level for the at least one antibiotic for the at least one microbe; identifying, during the administering, at least one microbe within the localized infection; and switching at least one of the antibiotics to a different antibiotic after identifying the at least one microbe.

The present disclosure further provides a method of treating a localized infection in a human patient by locally administering to the human patient a first antibiotic combination comprising two or more antibiotics, identifying at least one microbe within the localized infection, and switching at least one of the antibiotics to provide a second antibiotic combination, administering to the human patient the second antibiotic combination comprising two or more antibiotics. The second antibiotic combination results in an increased log₁₀ reduction (Log R) of the identified at least one microbe as compared to the first antibiotic combination against the identified microbe.

### DESCRIPTION OF FIGURES

These and other aspects will now be described in detail with reference to the following figures.

FIG. 1 is a table of generally accepted susceptibility limits of common antimicrobial agents for Staphylococcus aureus.

FIG. 2 illustrates the generation and adhesion of biofilm formed by certain bacterial and fungal pathogens.

FIG. 3A is a graphical representation of the minimum biofilm eradication concentration for tobramycin sulfate for common bacterial strains found in periprosthetic joint infections.

FIG. 3B is a graphical representation of the minimum biofilm eradication concentration for vancomycin HCl for common bacterial strains found in periprosthetic joint infections. FIG. 3C is a graphical representation of the minimum biofilm eradication concentration for vancomycin HCl and tobramycin sulfate for common bacterial strains found in periprosthetic joint infections.

FIG. 4 shows the average relative elution of vancomycin over time from commercially available bone cement typically used to stabilize the bones during a two-stage exchange treatment for periprosthetic joint infection.

FIG. 5 is a table of recommended safe dose and systemically administered concentrations of select antibiotics.

FIG. 6 is a graph of serum concentrations of vancomycin HCl in human patients during a 7-day treatment that included cyclic local irrigation of vancomycin HCl.

FIG. 7A shows an example of a treatment delivery system and kit.

FIG. 7B shows a detailed view of the treatment delivery system and kit of FIG. 7A.

### DETAILED DESCRIPTION

The disclosed subject matter relates to locally administered antibiotics at high concentrations that may improve treatment of wounds including localized infections and more specifically infections including medical devices such as joint prostheses or reconstructive implants. The dosing and method of safely administering high doses and concentrations of antibiotics is disclosed herein. The method of administering doses and concentrations of antibiotics greater than previously practiced levels of systemic therapy, while maintaining systemic exposure of the patient to levels at less than or equal to safe systemic concentration limits is also disclosed.

Locally administered antimicrobial therapies are not optimized for controlled delivery, partly due to a lack of medical devices to administer such therapy safely and effectively and partly due to a lack of research and development specifically intended to maximize local antimicrobial efficacy and minimize the risks associated with exposure of the wound to concentrated therapeutics and the environment during therapy. Recent advances in devices to irrigate wounds, specifically cyclic instillation and aspiration of wounds with saline, provide new opportunities for a previously unachievable method for safe and effective local delivery of therapeutic agents such as antibiotics by an irrigation route of administration.

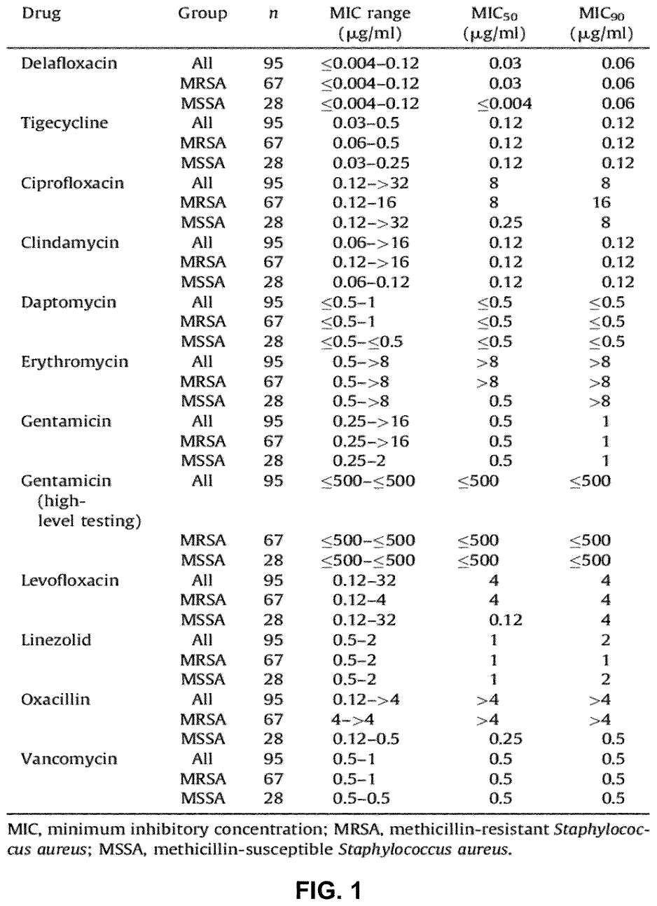

Successful eradication of infection by antimicrobial therapy is dependent upon the specific antimicrobial agent used, the specific pathogen, its susceptibility or resistance to the antimicrobial agent and the exposure of the pathogen to the antimicrobial agent, including the dose, concentration and duration of exposure, which are prescribed based on the mechanism of action and pharmacodynamic target of the antimicrobial agent. Susceptibility of pathogens in the clinical setting is determined by exposing a sample of the pathogen taken from the patient to concentrations within the therapeutic range of various antimicrobial agents and confirming effective eradication of the pathogen in a clinical laboratory test. The lowest antimicrobial concentration at which the specific pathogen is susceptible to a specific antimicrobial is known as the minimum inhibitory concentration (MIC). Pathogens that are not eliminated by exposure to an antimicrobial agent within its therapeutic range or clinically achievable concentrations are considered resistant to that antimicrobial agent. Pathogens may be resistant to a single agent, multi-drug resistant (MDR), extensively drug resistant (XDR), i.e., resistant to all antimicrobials except those from 2 or fewer classes or pan-drug resistant (PDR). The generally accepted susceptibility limits of common antimicrobial agents for Staphylococcus aureus are listed in FIG. 1. From the data in FIG. 1, it may be seen that the MIC at which a resistant strain of an organism (MRSA) is susceptible to an antimicrobial agent may be 2 to 10 times greater than the MIC of the susceptible strain of the same organism (MSSA). It may also be seen that the MIC of a specific antibiotic may be 1-2 orders of magnitude greater than the MIC of another antibiotic for the same strain of organism. MIC values are reported for the susceptibility of organisms in a planktonic (free floating) state. Typical breakpoint MICs used in clinical practice do not consider the possibility of the presence of a bio-film and inherent increase in associated MIC.

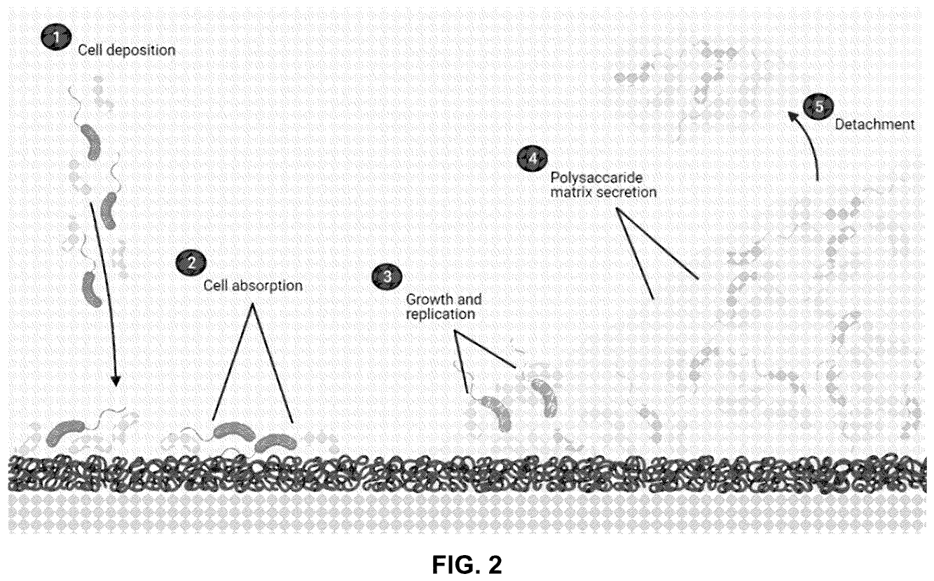

The biofilm formation process is shown in FIG. 2. A biofilm may be formed by a single bacterial species, but biofilms commonly consist of rich mixtures of many species of bacteria, as well as fungi, algae, protozoa and other microorganisms. In polymicrobial infections involving both bacteria and fungi a synergistic effect has been reported in which the antimicrobial resistance of the combined biofilm is greater than the that of the biofilm formed separately by either pathogen. Biofilms are held together by polysaccharide molecular strands, collectively termed “extracellular polymeric substances” or “EPS.” The cells produce EPS and are held together by these strands, allowing them to develop complex three-dimensional, resilient, attached communities. Biofilms may be as thin as a few cell layers or many inches thick, depending on environmental conditions.

Free-floating cells utilize nutrients, but do not have sufficient metabolic activity to deplete substrates from the neighborhood of the cells. In contrast, the collective metabolic activity of groups of cells in the biofilm leads to substrate concentration gradients and localized chemical microenvironments. Protective stress responses are effectively implemented in some of the cells in a biofilm at the expense of other cells which are sacrificed. The collective neutralizing power of groups of cells also leads to slow or incomplete penetration of antimicrobials in the biofilm.

Persister cells, i.e., antibiotic tolerant bacteria, accumulate and are physically retained by a biofilm matrix. Persister cells arise due to a state of dormancy, defined here as a state in which cells are metabolically inactive or anaerobic, and comprise a subpopulation of bacteria that become highly tolerant to antibiotics. Persister cells in biofilms appear to be responsible for the recalcitrance of chronic infections, since antibiotics kill the majority of cells; however, persister cells remain viable and repopulate biofilms when the level of antibiotics drops. Based on decades-old research, persister cells are thought to be less sensitive to antibiotics, because the cells are dormant and not undergoing cellular activities that antibiotics may corrupt or are anaerobic, which results in tolerance (i.e., no growth and slow death). This antibiotic tolerance occurs in the biofilms of members of many different genera, including Escherichia coli, Pseudomonas aeruginosa, Staphylococcus aureus, Methicillin resistant Staphylococcus aureus (MRSA), Vancomycin Resistant Enterococcus faecalis (VRE), Staphylococcus epidermidis, Lactobacillus acidophilus, and Gardnerella vaginalis.

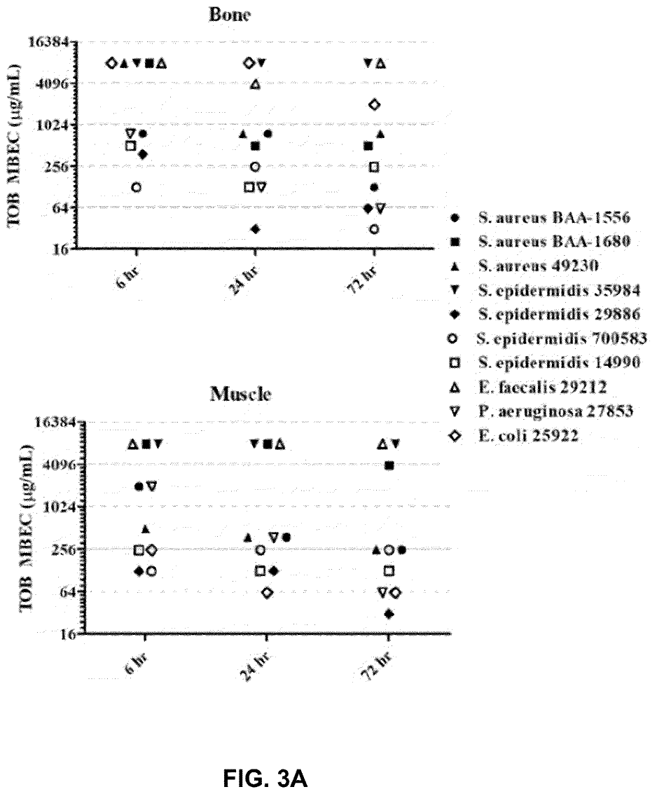

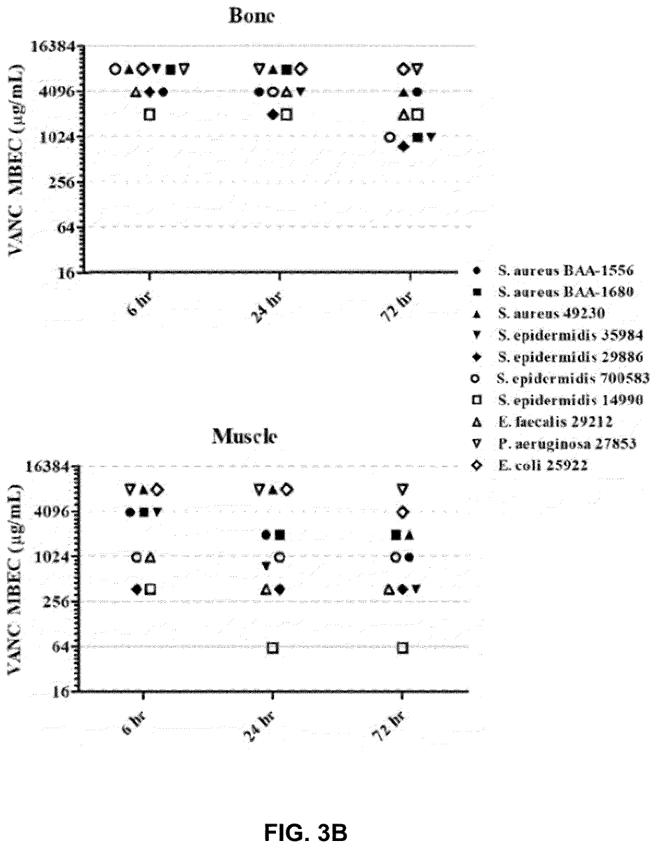

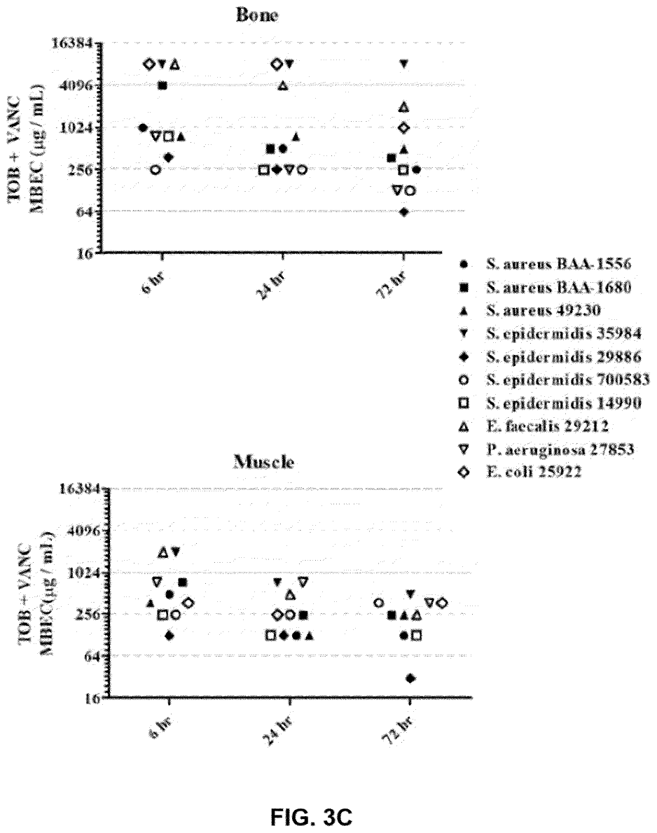

The formation of biofilm, a frequent occurrence in infected wounds with a medical device in situ such as PJI, BII, or FRI, begins early after onset of infection and provides an additional mechanism of resistance to antibiotic therapy. It is well established that the organisms in a mature biofilm structure, formed in a matter of days or a few weeks, are not susceptible to common antibiotics at the typical MIC of planktonic counterparts. However, significantly increased antimicrobial concentrations may be cidal to a pathogen in a mature biofilm, which is known as the biofilm bactericidal concentration (BBC) and defined as a 99.9% reduction in colony forming units (CFU's). The concentration at which eradication of an organism in a biofilm is accomplished is known as the minimum biofilm eradication concentration (MBEC) for the specific antimicrobial agent and organism tested. MBEC is not the same for all antimicrobial agents used to treat the same organism, nor is it the same for one antimicrobial agent applied to different strains or organisms. The MBEC for antibiotics most often used to treat common PJI organisms may be 100-1000× greater than the MIC for the same pairing of antibiotic and organism. The MBEC of tobramycin for common PJI organisms is shown in FIG. 3A. The MBEC of vancomycin for common PJI organisms is shown in FIG. 3B. The MBEC of vancomycin and tobramycin at 1:1 ratio for common PJI organisms is shown in FIG. 3C. This laboratory study shows that clinically occurring organisms in a mature biofilm structure exhibit MBEC levels that far exceed those achievable by administration of systemic antibiotics (intravenous or oral), as the MBEC far exceeds safe systemic concentrations of the antibiotics.

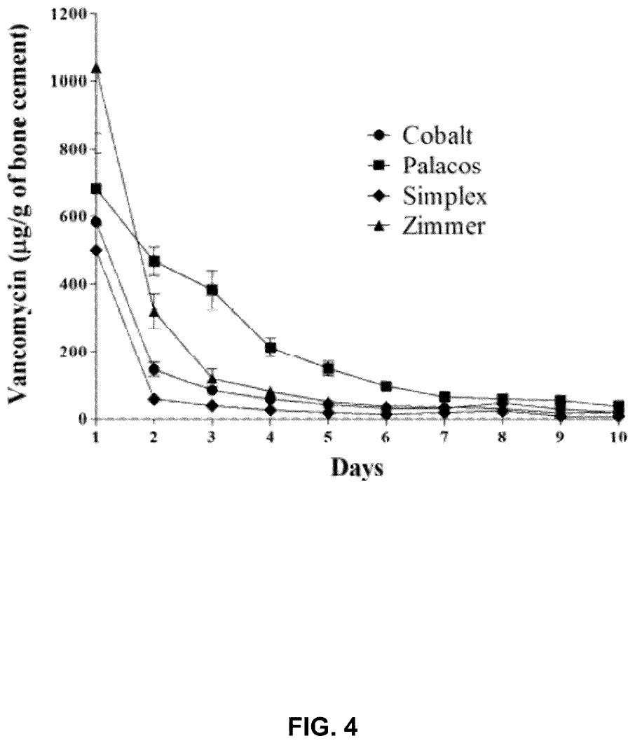

The standard of care for the treatment of chronic PJI is the two-stage exchange arthroplasty procedure, which begins with a surgical resection of the infected implant, aggressive debridement of bone and soft tissue, placement of a temporary implant that has been formed from antibiotic impregnated polymethylmethacrylate (PMMA), followed by systemic antibiotic therapy. The antibiotics in the PMMA elute into the surrounding tissue, with concentration of the eluting antibiotics decreasing exponentially over time and with distance from the temporary implant. The objective of the surgical resection is to dramatically reduce the bioburden of the infection, such that local antibiotic elution and concurrent systemic antibiotic therapy enable the patient's immune system to eradicate any remaining bacteria. An example of the antibiotic elution rate from a commercially available PMMA device is shown in FIG. 4. Elution rates vary widely in clinical practice due to differences in elution properties of available PMMA materials and to substantial differences in the mixing methods and dose of antibiotics mixed into PMMA temporary implants by surgeons. Alternative materials to PMMA, such as calcium sulfate, have also been used as locally eluting antibiotic carriers. In localized biofilm infections where the structural support of a temporary implant is not required, such as BII or FRI, the two-stage exchange procedure may be performed without any intermediary implant between the two surgical procedures.

The therapeutic effectiveness of antibiotics eluting from the temporary implant is limited to only tissue in sufficient proximity to the temporary implant to achieve a therapeutic concentration of the antibiotics and only for a brief time from implantation of the temporary implant to the time at which the local tissue concentration drops below therapeutic levels. Based upon the local bioburden, nature of the biofilm that remains following surgical debridement and the minimum biofilm eradication concentration of the antibiotic selected, the locally eluted antibiotic therapy may not be sufficient. Commercial products employing this technique are cleared for sale in the United States only as adjunctive to systemic antibiotic therapy. Clinically successful treatment of PJI (replacement of the infected prosthesis without reinfection, reoperation or administration of suppressive antibiotics) following an attempted two-stage exchange arthroplasty using the local antibiotic elution method have only been reported in about 50% of cases, 1 year after the first surgical treatment. The local antibiotics are absorbed into the tissue and circulatory system where they accumulate in addition to concurrently administered systemic antibiotics. Two-stage exchange arthroplasty patients are also at risk of severe nephrotoxicity and ototoxicity, due to initially very high concentrations of toxic antibiotics in the PMMA device, which cannot be adjusted without surgical intervention to explant the device. PMMA is also structurally weakened with increasing volume of antibiotics in the mixture. These toxicologic and mechanical considerations limit the maximum local concentration that may be delivered clinically by antibiotic eluting temporary implants.

Another limitation of antibiotic impregnated carriers is that they do not allow for optimization of pharmacodynamic target attainment. (e,g., aminoglycosides Tobramycin/Gentamicin) have a target of Pk concentration over MIC with a limited duration of exposure reaching maximum log kill after ~2 h, after which adaptive resistance and toxicity risks increase. Not allowing for elimination and constantly exposing pathogens to subinhibitory concentrations increases chance of adaptive resistance.

Local instillation of an antibiotic in solution for the treatment of PJI has been reported using repeated doses of antibiotics delivered via catheter to the periprosthetic tissue or joint space following aggressive debridement and one-stage exchange of the prosthesis. However, because instillation is by definition unidirectional (none of the instilled antibiotic is aspirated or flushed from the site of infection), and the unidirectional instillation doses are absorbed in the tissue and bloodstream, systemic toxicity considerations limit the local dose and concentration that may be safely administered by instillation. Whiteside et al. reported that instillation of very high concentrations of vancomycin (50,000-100,000 mcg/mL) once or twice daily into the joint space resulted in serum concentrations exceeding safe limits in multiple patients, which required a reduction in the instilled dose and concentration. Although the peak and trough concentrations of vancomycin in the joint space exceeded serum concentrations and were sustained above MIC of vancomycin for common organisms susceptible to vancomycin, the sustained local concentration was limited by exceeding safe systemic concentrations and was not maintained at or above MBEC of vancomycin (greater than 4,000 mcg/mL) for multiple common PJI organisms. Similarly, the maximum daily dose provided by this instillation method was 1,000 mg.

Both elution from an antibiotic impregnated carrier and localized instillation of antibiotic in solution carry a risk for localized tissue toxicity and even tissue necrosis, due to uncontrolled localized accumulation of antibiotic.

In one embodiment, the present invention is a significant improvement and departure over the prior art, because it comprises one or more doses of at least one antibiotic delivered via irrigation (instillation and subsequent aspiration) to a localized infection and/or biological tissue (e.g., periprosthetic tissue or joint space, breast capsule) of the human patient in need thereof. As such, in one embodiment, one or more doses of at least one antibiotic at or above BBC or MBEC delivered to the localized infection and/or biological tissue of the human patient in need thereof may be bidirectional or multidirectional, instead of unidirectional. In another aspect, the present invention is an improvement over the prior art by providing antibiotic concentrations and doses administered locally that exceed safe systemic concentrations and doses, but do not provide unsafe systemic blood plasma concentration levels to the human patient. Thus, the present invention provides methods for maintaining the concentration of at least one antibiotic at or above BBC or MBEC (e.g., for vancomycin, greater than 4,000 mcg/mL) for multiple common PJI organisms, and at 1 or more, 2 or more, 3 or more, 4 or more, 5 or more, 6 or more, 7 or more, 8 or more, 9 or more, 10 or more, 11 or more, or 12 or more times a day at the site of localized infection and/or biological tissue of the human patient. Similarly, the present methods allow for administering a greater total daily dose at least one antibiotic than what is permitted for systemic administration (e.g., maximum daily dose provided by this local administration method may be ≥3,000 mg/day, ≥4,000 mg/day, ≥5,000 mg/day, ≥6,000 mg/day, ≥7,000 mg/day, ≥8,000 mg/day, or ≥9,000 mg/day of vancomycin or vancomycin HCl or ≥100 mg/day, ≥200 mg/day, ≥300 mg/day, ≥400 mg/day, ≥500 mg/day, ≥750 mg/day, ≥1,000 mg/day of tobramycin or tobramycin sulfate).

From the examples above, it is clear that the effectiveness of elution and instillation methods of the prior art are limited to the organisms which are susceptible to the sustained available concentrations of the antibiotic delivered and attain the pharmacodynamic target. However, musculoskeletal infections are often polymicrobial and biofilm forming, and identification of the pathogen(s) typically occurs using tissue cultures 7-14 days after initiating therapy. In a significant minority of cases, the pathogens are never identified by tissue culture methods, and recent research employing genetic sequencing techniques have identified multiple organisms both in culture negative cases and cases with a single pathogen identified by tissue culture. So, local elution or instillation of a single antibiotic at a safe dose and concentration will frequently be ineffective at eradicating one or more organisms in an established biofilm, and the antibiotic cannot be tailored to the organisms, which are often not known at the time of antibiotic administration.

Broad spectrum systemic antibiotic therapy is the standard first-line treatment of localized infections, particularly when the pathogen is not yet identified or may be polymicrobial. In the case of infections in the presence of an implanted device such as PJI, BII, or FRI, broad spectrum therapy exceeding the BBC or MBEC for all identified or potential pathogens is preferred. Pre-clinical research to quantify BBC and MBEC of antibiotics for the pathogens found in local infections of humans has not been comprehensive. The number of potential antibiotics and known pathogens is large, and the BBC and MBEC values identified to date generally far exceed safe systemic dosing and concentrations of the antibiotics tested. In many reports of testing to establish MBEC values, no concentration of antibiotic tested was found to eradicate a biofilm of a specific organism, such that the MBEC for the specific antibiotic and strain of organism tested remains unknown.

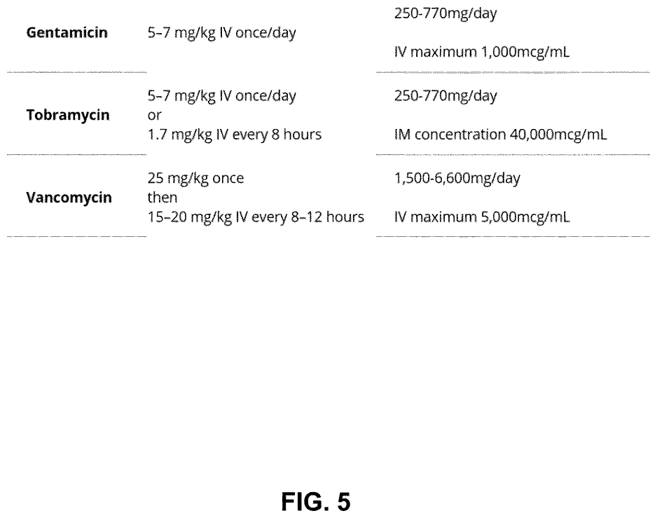

Broad spectrum antibiotic therapy for localized infections commonly includes a glycopeptide (e.g., vancomycin HCl) for coverage of gram+bacteria and an aminoglycoside (e.g., gentamicin or tobramycin sulfate) for coverage of gram−bacteria. Recommended safe dosing and systemic concentrations of these antibiotics are shown in FIG. 5.

As a route of administration, irrigation is defined as administration to bathe or flush open wounds or body cavities. By controlling dose, concentration and duration of exposure (bathing or flushing) time, a novel method of treating localized infections in a human patient has been developed. However, the irrigation of vancomycin and tobramycin within manufacturer's recommended dose and concentration does not provide local concentrations of antibiotics at or exceeding MBEC for multiple common PJI organisms. Moreover, the dose and concentration to achieve MBEC for biofilm forming organisms varies depending upon the organism, the maturity of the biofilm, the location of the biofilm, the implant or tissue to which the biofilm has attached, the dilution and absorption rate of the antibiotic at the site of irrigation and other factors. It should be noted that musculoskeletal infections may be mono- or polymicrobial in nature. In polymicrobial infections, the pathogens involved may include Gram+ and Gram-bacteria, fungi or any combinations of these pathogens. In such cases, as well as in cases of culture negative infections (i.e., no pathogen identified), it is best practice to administer more than one antimicrobial agent for the broadest possible coverage. What is needed clinically is a systemically safe antibiotic therapy which provides localized broad spectrum biofilm eradication under such highly variable clinical conditions.

In one aspect, provided herein are methods of treating a local infection in a biological tissue of a human patient in need thereof. The novel method described herein includes sustained administration of one or more antibiotics by an irrigation route of administration, at a dose and/or concentration exceeding previously established safe systemic dose and/or concentrations of the antibiotic(s), while providing safe systemic (serum) concentrations of the antibiotics. In at least one aspect, the novel method described herein includes sustained administration of one or more antibiotics by an irrigation route of administration at a dose and/or concentration at least equal to or exceeding the MBEC (e.g., ≥100% of the MBEC, ≥105% of the MBEC, ≥110% of the MBEC, ≥125% of the MBEC, ≥150% of the MBEC, ≥175% of the MBEC, ≥200% of the MBEC, ≥ 300% of the MBEC, ≥400% of the MBEC, ≥500% of the MBEC, ≥600% of the MBEC, ≥700% of the MBEC, ≥800% of the MBEC, ≥900% of the MBEC, ≥1,000% of the MBEC) while providing safe systemic (serum) concentrations of the one or more antibiotics.

In some embodiments, the method may include locally administering to the human patient a total daily dose of at least one antibiotic that substantially exceeds the maximum recommended daily systemic dose of the at least one antibiotic and providing a peak or trough serum concentration less than or equal to a maximum safe level for serum concentration of the at least one antibiotic.

The local infection may be monomicrobial, polymicrobial, or include an unidentified pathogen. In various aspects, the local infection may include, but is not limited to Staphylococcus aureus, methicillin resistant Staphylococcus aureus (MRSA), vancomycin resistant Enterococcus faecalis (VRE), Staphylococcus epidermidis, Enterococcus, Escherichia coli, Pseudomonas aeruginosa, undiagnosed/unidentified, or a combination thereof. The local infection may be inside or adjacent to a wound, a surgical site or incision, a joint replacement, a breast implant, or a combination thereof. Various antibiotics used to treat infections and biofilms may be used with the disclosed methods. Non-limiting examples of antibiotics include aminoglycosides, glycopeptides, cyclic lipopeptides, vancomycin, tobramycin, gentamycin, daptomycin and combinations thereof.

The daily dose may comprise a single administration or a plurality of administrations during the day. For example, the daily dose may be administered as 1, 2, 3, 4, 5, 6, 7, 8, 9, 10, 11, 12, 13, 14, 15, 16, 17, 18, 19, 20, 21, 22, 23, or 24 administrations during the day. In some examples, the daily dose may be administered over at least 1, at least 2, at least 5, at least 10, at least 15, at least 20, at least 21, at least 21.5, or at least 22 administrations. The administrations may be every 30 minutes or less, about every hour, about every 2 hours, about every 3 hours, about every 4 hours, about every 5 hours, about every 6 hours, about every 7 hours, about every 8 hours, about every 9 hours, about every 10 hours, about every 12 hours, about every 18 hours, or about every 24 hours.

The antibiotics may be locally administered by irrigating the biological tissue with the at least one antibiotic one or more times. Irrigating the biological tissue includes soaking the biological tissue with the at least one antibiotic and subsequently aspirating the at least one antibiotic from the biological tissue after a period of time. Surprisingly, this allows for the local concentration at the infection site to be maintained at a high concentration without being systemically absorbed at the high concentrations. This provides high local MBEC concentrations to be reached at the infection site to eradicate the infection/biofilm while keeping serum concentrations below recommended limits so the patient is not adversely affected.

Irrigating the biological tissue may include soaking the biological tissue with the antibiotic for about 30 minutes to about 180 minutes, about 30 minutes to about 45 minutes, about 30 minutes to about 60 minutes, about 60 minutes to about 90 minutes, about 60 minutes to about 120 minutes, or about 120 minutes to about 180 minutes. In various examples, irrigating biological tissue may include soaking with the antibiotic for 30 minutes, 60 minutes, 90 minutes, 120 minutes, 150 minutes, or 180 minutes.

The volume of administered antibiotic may be sufficient to allow all the biological tissue to be exposed throughout the soak period. The volume of the administered dose for each soaking step may be about 20 cc, about 30 cc, about 40 cc, about 50 cc, about 60 cc, about 70 cc, about 80 cc, about 90 cc, about 100 cc, or greater than 100 cc.

The irrigating may be performed at least once daily, at least twice daily, at least 5 times daily, at least 10 times daily, at least 15 times daily, at least 20 times daily, at least 21 times daily, or at least 22 times daily. In one example, an antibiotic may be irrigated at the infection site every hour for 20 to 22 hours. In some examples, two or more antibiotics may be irrigated at the infection site in the biological tissue. The two or more antibiotics may be irrigated at different frequencies. For example, one antibiotic may be irrigated at the infection site hourly for 12 to 24 hours and a second antibiotic may be irrigated at the infection site 1 to 2 times per day. The first antibiotic may be aspirated from the infection site before the second antibiotic is installed/soaked. Alternatively, two or more antimicrobial agents, including antibiotics may be irrigated simultaneously from a single solution source or separate solution sources.

In some embodiments, the method may further include irrigating the localized infection and/or biological tissue with a rinsing liquid (e.g., saline) one or more times between antimicrobial administrations. In some aspects, the rinsing liquid may be sterile saline or water. Rinsing the infection site may further assist in reducing bioburden or clearing the previously administered antibiotic to allow for a subsequent high dose administration.

The daily dose of the at least one antibiotic may substantially exceed the maximum recommended daily systemic dose of the at least one antibiotic. Substantially exceeding the maximum recommended daily systemic dose of the at least one antibiotic may include the daily dose exceeding the maximum recommended daily systemic dose of the at least one antibiotic by at least 125%, at least 150%, at least 175%, at least 200%, at least 225%, at least 250%, at least 275%, at least 300%, at least 400%, at least 500%, at least 600%, at least 700%, at least 800%, at least 900%, at least 1,000% or greater.

In some embodiments, the at least one antibiotic is vancomycin. The locally administered concentration of vancomycin may be at least 3,000 mcg/mL, at least 5,000 mcg/mL, at least 7,000 mcg/mL, at least 10,000 mcg/mL, at least 20,000 mcg/mL, at least 40,000 mcg/mL, or at least 50,000 mcg/mL. In various examples, the locally administered concentration of vancomycin may be greater than 2,500 mcg/mL, about 18,333 mcg/mL, about 34,167 mcg/mL, or about 50,000 mcg/mL. In an example, the maximum recommended daily systemic dose of vancomycin is 60 mg/kg/day which may be about 1,200 to 7,500 mg/day for 95% of the U.S. population. Therefore, the locally administered daily dose of vancomycin may be about 3,000 to about 50,000 mg/day. In various examples, the locally administered daily dose of vancomycin is at least 3,500 mg/day, at least 4,000 mg/day, at least 5,000 mg/day, at least 6,000 mg/day, at least 7,000 mg/day, at least 8,000 mg/day, at least 9,000 mg/day, at least 10,000 mg/day, at least 12,000 mg/day, at least 14,000 mg/day, at least 16,000 mg/day, at least 18,000 mg/day, at least 20,000 mg/day, at least 30,000 mg/day, at least 40,000 mg/day, at least 50,000 mg/day, or greater than 50,000 mg/day. In at least one example, the locally administered daily dose of vancomycin may be about 9,450 mg/day. In this example, each local dose may be about 450 mg (e.g. 450 mg of vancomycin in a saline solution (0.9%)) when administered 21 times per day.

The total dose of vancomycin administered over all administration days may range from about 15 g to 100 g, about 15 g to 20 g, about 20 g to 25 g, about 25 g to 30 g, about 30 g to 35 g, about 35 g to 40 g, about 40 g to 45 g, about 45 g to 50 g, about 50 g to 60 g, about 60 g to 70 g, about 70 g to 80 g, about 80 g to 90 g, or about 90 g to 100 g. For longer courses of therapy, the total dose of vancomycin administered over all administration days may be about 100 g to 200 g, 100 g to 300 g, or about 200 g to 400 g or greater.

Although a high concentration is administered locally, the benefit of the disclosed methods is that the serum concentration for the antibiotics is maintained at a level below the maximum safe level. Therefore, the patient receives the benefit of high concentrations of the antibiotic (e.g., above the BBC or MBEC level) to treat the infection while maintaining safe serum levels and minimizing any side effects to the patient. The maximum safe trough level for serum concentration of vancomycin may be about 30 mcg/mL or about 25 mcg/mL. In some examples, the trough serum concentration of vancomycin when administered according to the disclosed methods is less than 30 mcg/mL, less than 25 mcg/mL, less than 20 mcg/mL, less than 15 mcg/mL, less than 10 mcg/mL, or less than 7 mcg/mL.

In some embodiments, the at least one antibiotic is tobramycin. The locally administered concentration of tobramycin may be at least 2,000 mcg/mL, at least 3,000 mcg/mL, at least 4,000 mcg/mL, at least 5,000 mcg/mL, at least 6,000 mcg/mL, at least 7,000 mcg/mL, at least 8,000 mcg/mL, at least 10,000 mcg/mL, at least 12,000 mcg/mL, at least 15,000 mcg/mL, at least 20,000 mcg/mL, at least 25,000 mcg/mL, at least 30,000 mcg/mL, at least 35,000 mcg/mL, or at least 40,000 mcg/mL. In some examples, the locally administered concentration of tobramycin may be greater than 1,600 mcg/mL, about 14,400 mcg/mL, about 27,200 mcg/mL, or about 40,000 mcg/mL. In an example, the maximum recommended daily systemic dose of tobramycin may be about 10 mg/kg/day, which may be about 200 to about 1,250 mg/day in 95% of the U.S. population. In various examples, the locally administered daily dose of tobramycin is at least 100 mg/day, at least 200 mg/day, at least 400 mg/day, at least 600 mg/day, at least 800 mg/day, at least 1,000 mg/day, at least 1,200 mg/day, at least 1,400 mg/day, at least 1,600 mg/day, at least 2,000 mg/day, or greater than 2,500 mg/day. In at least one example, the locally administered daily dose of tobramycin may be about 450 mg/day. In this example, each local dose may be about 450 mg (e.g., 450 mg of tobramycin in a saline solution (0.9%)) when administered once per day.

The total dose of tobramycin administered over all administration days may range from about 1 g to 2 g, about 2 g to 3 g, about 3 g to 4 g, about 4 g to 6 g, about 6 g to 8 g, or about 8 g to 10 g. For longer courses of therapy, the total dose of tobramycin administered over all administration days may be about 10 g to 15 g or about 15 g to 20 g or greater.

The maximum safe level for peak serum concentration of tobramycin may be less than 10 mcg/mL. In some examples, the peak serum concentration of tobramycin is less than less than 9 mcg/mL, less than 8 mcg/mL, less than 7 mcg/mL, less than 6 mcg/mL, less than 5 mcg/mL, or less than 3 mcg/mL.

In some embodiments, the at least one antibiotic is daptomycin. The locally administered concentration of daptomycin may be at least 3,000 mcg/mL, at least 5,000 mcg/mL, at least 7,000 mcg/mL, at least 10,000 mcg/mL, at least 20,000 mcg/mL, at least 40,000 mcg/mL, or at least 50,000 mcg/mL. In various examples, the locally administered concentration of daptomycin may be greater than 2,500 mcg/mL, about 18,333 mcg/mL, about 34,167 mcg/mL, or about 50,000 mcg/mL. In an example, the locally administered daily dose of daptomycin may be about 3,000 to about 50,000 mg/day. In various examples, the locally administered daily dose of daptomycin is at least 3,500 mg/day, at least 4,000 mg/day, at least 5,000 mg/day, at least 6,000 mg/day, at least 7,000 mg/day, at least 8,000 mg/day, at least 9,000 mg/day, at least 10,000 mg/day, at least 12,000 mg/day, at least 14,000 mg/day, at least 16,000 mg/day, at least 18,000 mg/day, at least 20,000 mg/day, at least 30,000 mg/day, at least 40,000 mg/day, at least 50,000 mg/day, or greater than 50,000 mg/day.

The total dose of daptomycin administered over all administration days may range from about 15 g to 100 g, about 15 g to 20 g, about 20 g to 25 g, about 25 g to 30 g, about 30 g to 35 g, about 35 g to 40 g, about 40 g to 45 g, about 45 g to 50 g, about 50 g to 60 g, about 60 g to 70 g, about 70 g to 80 g, about 80 g to 90 g, or about 90 g to 100 g. For longer courses of therapy, the total dose of daptomycin administered over all administration days may be about 100 g to 200 g or about 200 g to 400 g or greater.

In an example, to accomplish local concentrations at MBEC and safe serum levels for multiple organisms with vancomycin, vancomycin concentrations of up to 10,000 mcg/mL may be administered by local irrigation hourly with 30-minute soak and aspiration cycles while maintaining trough serum concentrations of vancomycin below the maximum safe level of 30 mcg/mL. Tobramycin concentrations of up to 6,400 mcg/mL may be administered by local irrigation once or twice daily with 120-minute soak and subsequent aspiration while maintaining peak serum concentrations of tobramycin below the maximum safe level of 10 mcg/mL and trough serum concentration ≤2 mcg/mL. Toxicity is associated with sustained trough levels >2 mcg/mL. The specific concentration and daily dose may be varied while maintaining safe serum concentrations by administering the antibiotics for shorter or longer soak and aspiration cycles (resulting in shorter or longer exposure times locally and less or more systemic absorption and accumulation). The concentration and daily dose may be substantially greater during the initial period of treatment to maximize initial the biocidal effect and reduced for the balance of the treatment time. In some examples, initial concentration and daily doses are twice that of the remaining doses, are 3 times, 5 times, 10 times or 20 times that of the remaining doses.

In some embodiments, the method includes locally administering two antibiotics or two or more antibiotics. The two antibiotics may be sequentially administered or not administered simultaneously. In other aspects, at least one dose of each antibiotic may be administered simultaneously. In further aspects, the two antibiotics may be administered at different frequencies. For example, a first antibiotic may be locally administered once daily where it soaks for 15 minutes to 4 hours, 30 minutes to 4 hours, 45 minutes to 4 hours, 1 hour to 4 hours, 15 minutes to 3 hours, 30 minutes to 3 hours, 45 minutes to 3 hours, 1 hour to 3 hours, 15 minutes to 2 hours, 30 minutes to 2 hours, 45 minutes to 2 hours, or 1 hour to 2 hours, and is subsequently aspirated over 30 minutes and a second antibiotic may be locally administered hourly for 10 to 15 hours, 15 to 20 hours, 20 to 22 hours, or 20 to 24 hours. The second antibiotic may be administered after the aspiration of the first antibiotic or the two antibiotics may be administered simultaneously when the first antibiotic is being administered.

The two antibiotics may be vancomycin and tobramycin, in some instances. In at least one example, the vancomycin may be locally administered hourly for 20 to 22 hours, or 20 to 24 hours, where the vancomycin soaks for about 30 minutes and is subsequently aspirated over 30 minutes, while the tobramycin is locally administered once daily. The two antibiotics may be tobramycin and daptomycin, in other instances. In at least one example, the daptomycin may be locally administered hourly for 20 to 22 hours, or 20 to 24 hours, where the daptomycin soaks for about 15, 30, 45, or 60 minutes and is subsequently aspirated over 30 minutes, while the tobramycin is locally administered once daily.

In various embodiments, the local administration of the one or more antibiotics may be for 24 hours, 2 days, 3 days, 4 days, 5 days, 6 days, 7 days, 10 days or 14 days. The local administration of the high daily dose of the one or more antibiotics may allow for a reduction in the amount of time that the antibiotics need to be administered, as compared to intravenous administration, oral administration or lower concentration local administration.

When two or more antibiotics are administered, at least one of the antibiotics may be administered at a daily dose that substantially exceeds the maximum recommended daily systemic dose of that antibiotic. In some instances, both antibiotics may be administered at a daily dose that substantially exceeds the maximum recommended daily systemic dose of that antibiotic. For example, when vancomycin and tobramycin are administered, the concentration of the locally administered daily dose of vancomycin may range from about 3,000 mcg/mL to about 50,000 mcg/mL and the locally administered daily dose of tobramycin may range from about 2,000 mcg/mL to about 40,000 mcg/mL. When daptomycin and tobramycin are administered, the concentration of the locally administered daily dose of daptomycin may range from about 3,000 mcg/mL to about 50,000 mcg/mL and the locally administered daily dose of tobramycin may range from about 2,000 mcg/mL to about 40,000 mcg/mL.

The at least two antibiotics may be administered at a fixed dose ratio. For example, vancomycin/daptomycin and tobramycin may be administered at a fixed dose ratio of 5:1 to 40:1, 5:1 to 10:1, 10:1 to 15:1, 15:1 to 20:1, 20:1 to 25:1, 25:1 to 30:1, 30:1 to 35:1, or 35:1 to 40:1. In some examples, the fixed dose ratio of vancomycin/daptomycin to tobramycin may be 32.8:1, 22:1, or 11:1. The daily dose of vancomycin/daptomycin may be about 3,000 to 50,000 mg/day or the daily dose of tobramycin may be about 100 to 1,200 mg/day. In an example, the daily dose of vancomycin/daptomycin may be at least 3,000 mg/day and/or the daily dose of tobramycin may be at least 100 mg/day. In another example, the daily dose of vancomycin/daptomycin may be at least 4,000 mg/day and/or the daily dose of tobramycin may be at least 125 mg/day. In a further example, the daily dose of vancomycin/daptomycin may be at least 5,000 mg/day and/or the daily dose of tobramycin may be at least 150 mg/day. Other combinations of administration timing and doses are contemplated to effectively treat the specific infection and patent in need thereof.

The local administration of the at least two antibiotics via irrigation may maintain the peak or trough serum concentrations of each of the at least two antibiotics at or below a maximum safe serum concentration of each antibiotic. The maximum safe serum concentration may vary for each antibiotic and may vary by patient. In some examples, the maximum safe trough serum concentration for vancomycin or daptomycin may be 20 mcg/mL to about 30 mcg/mL and the maximum safe peak serum concentration for tobramycin may be about 4 mcg/mL to about 10 mcg/mL. In some examples, the trough serum concentration of vancomycin or daptomycin may be less than about 30 mcg/mL, less than about 25 mcg/mL, less than about 20 mcg/mL, or less than about 10 mcg/mL and the trough serum concentration of tobramycin may be less than about 10 mcg/mL, less than about 5 mcg/mL, or less than about 3 mcg/mL.

Further provided herein is a method of treating a local infection of a human patient by locally administering by irrigation to the human patient a local concentration of at least one antibiotic that exceeds the maximum recommended concentration of the at least one antibiotic such that the local administration by irrigation provides a peak or trough serum concentration that is less than the maximum safe serum concentration of the at least one antibiotic.

Also provided herein is a method of treating a musculoskeletal infection of a human patient by administering to the human patient at least one antibiotic at locally administered concentrations that exceeds the minimum biofilm eradication concentration (MBEC) of the at least one antibiotic for one or more of Staphylococcus aureus, methicillin resistant Staphylococcus aureus (MRSA), vancomycin resistant Enterococcus faecalis (VRE), Staphylococcus epidermidis, Enterococcus, Escherichia coli, and Pseudomonas aeruginosa. The administration of the at least one antibiotic at a concentration that exceeds the MBEC may provide a serum concentration of the antibiotic less than the maximum safe serum concentration of the antibiotic. In some embodiments, the method may include administering two antibiotics, where at least one of the two antibiotics is administered at a locally administered concentration that exceeds the MBEC of that antibiotic for one or more of Staphylococcus aureus, methicillin resistant Staphylococcus aureus (MRSA), vancomycin resistant Enterococcus faecalis (VRE), Staphylococcus epidermidis, Enterococcus, Escherichia coli, and Pseudomonas aeruginosa. In other embodiments, the method may include administering at least two antibiotics at locally administered concentrations that each exceed the MBEC of the at least one antibiotic for one or more of Staphylococcus aureus, methicillin resistant Staphylococcus aureus (MRSA), vancomycin resistant Enterococcus faecalis (VRE), Staphylococcus epidermidis, Enterococcus, Escherichia coli, and Pseudomonas aeruginosa. The peak or trough serum concentration for both antibiotics may be less than a maximum safe serum concentration for each antibiotic.

In various examples, the at least two antibiotics are vancomycin or daptomycin and tobramycin. The locally administered concentration of vancomycin or daptomycin may range from about 3,000 mcg/mL to about 50,0000 mcg/mL and the locally administered concentration of tobramycin may range from about 100 mcg/mL to about 1,200 mcg/mL. In an example, the locally administered concentration of vancomycin or daptomycin may be at least 6,000 mcg/mL and the locally administered concentration of tobramycin may be at least 200 mcg/mL. The peak serum concentration of tobramycin may be less than 10 mcg/mL and the trough serum concentration of vancomycin or daptomycin may be less than 30 mcg/mL.

Additionally provided herein is a method of treating a local infection of a human patient by locally administering to the human patient a daily dose of a first antibiotic that substantially exceeds the maximum recommended systemic daily dose of the first antibiotic and providing a peak or trough serum concentration less than or equal to a maximum safe serum concentration of the first antibiotic.

In an embodiment, the method may include locally administering to the human patient a daily dose of a first antibiotic and a daily dose of a second antibiotic that substantially exceeds the maximum recommended daily dose of the first antibiotic and the second antibiotic and providing a peak or trough serum concentration less than or equal to a maximum safe serum concentration of the first antibiotic and the second antibiotic. In some aspects, a maximum recommended daily dose of each antibiotic is the maximum recommended systemic daily dose.

In another embodiment, the method may include locally administering to the human patient a daily dose of at least two antibiotics that substantially exceeds the maximum recommended systemic daily dose of each of the at least two antibiotics and providing a peak or trough serum concentration less than or equal to a maximum safe serum concentration of each of the at least two antibiotics. In some aspects, a maximum recommended daily dose of each antibiotic is the maximum recommended systemically administered daily dose.

Further disclosed herein is a method of treating a local infection in a biological tissue of a human patient by locally administering to the human patient a dose of at least one antibiotic that exceeds 3,500 mg/day. In some examples, the at least one antibiotic is vancomycin or daptomycin and the locally administered dose is at least 3,500 mg/day, at least 4,000 mg/day, at least 5,000 mg/day, at least 6,000 mg/day, at least 7,000 mg/day, at least 8,000 mg/day, at least 9,000 mg/day, or at least 10,000 mg/day.

Also disclosed herein is a method of treating a local infection in a biological tissue of a human patient by locally administering to the human patient a dose of tobramycin that exceeds 150 mg/day. In various examples, the locally administered dose is at least 200 mg/day, at least 300 mg/day, at least 400 mg/day, or at least 500 mg/day.

In a method of treating a local infection in a biological tissue of a human patient, the method may include locally administering to the human patient two or more doses of at least one antibiotic within 12 to 24 hours at a concentration that exceeds the MBEC level for the at least one antibiotic for the local infection. In an example, two to five doses of the at least one antibiotic may be locally administered within 12 hours or 24 hours at a concentration that exceeds the MBEC level for the at least one antibiotic for the local infection. In another example, three to seven doses of the at least one antibiotic may be locally administered within 12 hours or 24 hours at a concentration that exceeds the MBEC level for the at least one antibiotic for the local infection. In yet another example, eight to twelve doses of at least one antibiotic may be locally administered within 12 hours or 24 hours at a concentration that exceeds the MBEC level for the at least one antibiotic for the local infection.

In various embodiments, the local administration according to any of the disclosed methods may result in a ≥3-log, ≥3.1-log, ≥3.2-log, ≥3.3-log, ≥3.4-log, ≥3.5-log, ≥3.6-log, ≥ 3.7-log, ≥3.8-log, ≥3.9-log, ≥4-log, ≥4.1-log, ≥4.2-log, ≥4.3-log, ≥4.4-log, ≥4.5-log, ≥4.6-log, ≥4.7-log, ≥4.8-log, ≥4.9-log, ≥5-log, ≥5.1-log, ≥5.2-log, ≥5.3-log, ≥5.4-log, ≥5.5-log, ≥ 5.6-log, ≥5.7-log, ≥5.8-log, ≥5.9-log, ≥6-log, ≥6.1-log, ≥6.2-log, ≥6.3-log, ≥6.4-log, ≥6.5-log, ≥6.6-log, ≥6.7-log, ≥6.8-log, ≥6.9-log, ≥7-log, ≥7.1-log, ≥7.2-log, ≥7.3-log, ≥7.4-log, ≥ 7.5-log, ≥7.6-log, ≥7.7-log, ≥7.8-log, ≥7.9-log, ≥8-log, or greater reduction in the local infection (e.g., Staphylococcus aureus, methicillin resistant Staphylococcus aureus (MRSA), vancomycin resistant Enterococcus faecalis (VRE), Staphylococcus epidermidis, Enterococcus, Escherichia coli, Pseudomonas aeruginosa, unidentified infection, or a combination thereof) within 7 days (e.g. after local administration for 7 days), within 5 days, within 3 days, or within 24 hours of administering the antibiotics. Stated differently, the local administration according to any of the disclosed methods may result in a log₁₀ reduction (Log R) in the local infection of 3, 3.5, 4, 4.5, 5, 5.5, 6, 6.5, or greater within 7 days (e.g. after local administration for 7 days), within 5 days, within 3 days, or within 24 hours of administering the antibiotics. In some examples, the local infection may be considered to be eradicated once there has been a 3-log reduction or greater in the infection. The local administration according to any of the disclosed methods may result in greater than 98%, 98.5%, 99%, 99.9%, 99.99%, 99.999%, or 100.0% of CFU's of the local infection are killed within 7 days, 5 days, 3 days, or 24 hours of administering.

The method disclosed herein can be used to provide personalized treatment to a patient. In some embodiments, a method of treating a localized infection in a human patient includes identifying at least one microbe within the localized infection, and locally administering to the human patient two or more antibiotics, where at least one of the antibiotics is administered at a concentration that exceeds a MBEC level for the at least one antibiotic for the at least one microbe. In one example, when the microbe is one or more of Staphylococcus aureus, Staphylococcus epidermidis, Enterococcus, Escherichia coli, and Pseudomonas aeruginosa, the two or more antibiotics are vancomycin and tobramycin. In another example, when the microbe is methicillin resistant Staphylococcus aureus (MRSA) or vancomycin resistant Enterococcus faecalis (VRE), the two or more antibiotics are daptomycin and tobramycin.

The identification of the at least one microbe can be used to determine which antibiotics to locally administer to the patient. The at least one microbe may be identified by laboratory testing of a sample from the site of the localized infection. In some instances, the microbe may be identified prior to administering the two or more antibiotics. In other instances, the microbe may not be identified or may be identified after the administering has already started. For example, it may take hours to days or days to weeks to receive the results for the identification of the microbe(s). Therefore, it may be in the interest of the patient to begin local administration of at least two antibiotics prior to knowing the identification of the microbe(s) and then switch one or more of the antibiotics during the course of administration if the microbe(s) identified would be more susceptible to a different antibiotic or different antibiotic combination of at least two antibiotics.

In some embodiments, a method of treating a localized infection in a human patient may include locally administering to the human patient two or more antibiotics, identifying, during the administering, at least one microbe within the localized infection, and optionally switching at least one of the antibiotics to a different antibiotic, after identifying the at least one microbe. In another embodiment, provided herein is a method of treating a localized infection in a human patient comprising locally administering to the human patient a first antibiotic combination comprising two or more antibiotics, identifying at least one microbe within the localized infection, and optionally switching at least one of the antibiotics to provide a second antibiotic combination comprising two or more antibiotics, where the second antibiotic combination results in an increased log₁₀ reduction (Log R) of the identified at least one microbe (e.g., an improvement of ≥ 0.5-log, ≥1-log, ≥1.5-log, ≥2.0-log, ≥2.5-log, ≥3.0-log, ≥3.5-log, ≥4.0-log, or greater improvement) as compared to the first antibiotic combination against the identified microbe (e.g., within 7 days, within 6 days, within 5 days, within 4 days, within 3 days, within 2 days, or within 24 hours of administering the second antibiotic combination). At least one of the antibiotics is administered at a concentration that exceeds a MBEC level for the at least one antibiotic for the at least one microbe and at least one of the antibiotics may be switched if the treatment would be more effective against the identified microbe. In one example, if the two or more antibiotics first administered are vancomycin and tobramycin, the administration may stay the same when the microbe is one or more of Staphylococcus aureus, Staphylococcus epidermidis, Enterococcus, Escherichia coli, and Pseudomonas aeruginosa, but the vancomycin may be switched to daptomycin when the microbe is methicillin resistant Staphylococcus aureus (MRSA), vancomycin resistant Enterococcus faecalis (VRE), or a combination thereof.

The methods disclosed herein may provide for decreased re-infection rates, decreased rates of readmission to the hospital, and/or decreased rates of mortality or sepsis as compared to patients treated without high local concentrations of antibiotics. In some examples, patients treated with the disclosed methods may have an at least 10%, at least 20%, at least 30%, at least 40%, or at least 50% lower re-infection rate than a patient not treated using the disclosed methods. In some examples, patients treated with the disclosed methods may have an at least 10%, at least 20%, at least 30%, at least 40%, or at least 50% lower readmission rate than a patient not treated using the disclosed methods. In additional examples, the patient treated may have no readmission for at least 30 days, at least 90 days, at least 180 days, or at least 1 year. In some examples, patients treated with the disclosed methods may have an at least 10%, at least 20%, at least 30%, at least 40%, or at least 50% lower mortality rate than a patient not treated using the disclosed methods. In some examples, patients treated with the disclosed methods may have an at least 10%, at least 20%, at least 30%, at least 40%, or at least 50% lower rate of sepsis or other infection than a patient not treated using the disclosed methods.

In another embodiment, locally administering to the human patient two or more doses of at least one antibiotic within 12 hours or 24 hours may maintain an AUC/MBEC at the biological tissue for at least 5 hours a day, at least 10 hours a day, at least 12 hours a day, at least 18 hours a day, or at least 22 hours a day.

In a further embodiment, the method may include administering by local irrigation to the human patient at least a 25 mg dose of vancomycin or daptomycin at a concentration of 3,000 mcg/mL or greater and providing less than a 30 mcg/mL trough serum concentration in the patient for 24 hours following administration.

In additional embodiments, the method may include administering by local irrigation to the human patient a dosage of vancomycin or daptomycin and tobramycin, where the dosage includes at least a 50 mg dose of vancomycin or daptomycin at a concentration of 3,000 mcg/mL or greater, and at least a 50 mg dose of tobramycin at a concentration of 1,500 mcg/mL or greater. The administering by local irrigation may then provide a trough serum concentration less than 30 mcg/mL of vancomycin or daptomycin and a peak serum concentration less than 10 mcg/mL of tobramycin in the patient for 24 hours following administration.

In one method, 50 cc each of vancomycin or daptomycin and tobramycin may be administered sequentially at equal concentrations of 3,000 mcg/mL for 7 days, with vancomycin or daptomycin administered hourly for 20 hours and tobramycin administered once daily, a fixed dose ratio of 20:1. The daily doses in this method are 150 mg tobramycin and 3,000 mg of vancomycin/daptomycin, and the total doses are 1.05 g of tobramycin and 21 g of vancomycin/daptomycin. In an alternate method, vancomycin/daptomycin may be administered hourly for 20 hours at 10,000 mcg/mL, twice the concentration of tobramycin at 5,000 mcg/mL once daily, a fixed dose ratio of 40:1 for a period of 5 days. The daily doses in this method are 250 mg tobramycin and 20,000 mg of vancomycin/daptomycin, and the total doses are 1.25 g of tobramycin and 100 g of vancomycin/daptomycin. In such methods, one or both the concentrations and the daily doses of the antibiotics administered exceed the previously known safe limits for administration of each antibiotic. In such methods, the novel doses and concentrations are administered while providing safe serum concentrations of each antibiotic at or below clinically established limits. The maximum doses and concentrations that may be administered are calculated pharmacokinetically based on the selected daily exposure and known values of pharmacokinetic parameters, and such doses and concentrations may be adjusted empirically prior to or during administration in the case of a patient with abnormal absorption or elimination of the antibiotic.

In one method, one or more antibiotics may be administered at a previously known safe dose in concentration(s) that exceed previously known safe concentrations of such antibiotic(s) while providing clinically established safe systemic levels of the antibiotic(s). This is accomplished by irrigation of the site of infection at higher concentrations less frequently per day, so as to administer a daily dose that is maintained within previously known safe limits. In an alternate method, one or more antibiotics may be administered in a daily dose that exceeds previously known safe daily doses in concentration(s) within known safe limits while providing clinically established safe systemic levels of the antibiotic(s). This is accomplished by irrigation of the site of infection at within previously known safe concentration limits more frequently per day so as to administer a daily dose that exceeds previously known safe limits.

When two or more antibiotics are administered in the methods described herein, they may be administered at a fixed dose ratio of 1:1; however, based on highly variable MBEC values of different antibiotics for the same organism, it may be beneficial to administer antibiotics at fixed dose ratios exceeding the maximum dose ratios of previously established safe daily doses of such antibiotics. For example, based on the 5th and 95th percentile of patient weight in the U.S., the minimum and maximum recommended daily systemic dose of vancomycin is about 1,200 g and 7,500 g respectively, and the minimum and maximum recommended daily systemic dose of tobramycin is about 200 g and 1250 g respectively. The resulting recommended fixed dose ratio of vancomycin to tobramycin is therefore 6:1. The methods disclosed herein describe administration of antibiotics at or exceeding MBEC for the target organisms while maintaining safe systemic levels of the antibiotics. In such cases, the fixed dose ratios may exceed previously known systemic dose ratios. In one method, the dose ratio of vancomycin to tobramycin is 40:1 as described above. In an alternate method, the fixed dose ratio of vancomycin to tobramycin is 125:1. In another alternate method, the fixed dose ratio of vancomycin to tobramycin is 10:1.

Not all antimicrobial agents may be administered simultaneously, due to incompatibility of the antibiotic solutions or differences in their pharmacodynamic targets. For example, tobramycin is administered for the optimal concentration dependent target with an optimal exposure of 2 hours at therapeutic levels, whereas vancomycin is administered with an optimal pharmacodynamic target of AUC/MIC or total daily exposure. It is a further benefit of an irrigation route of antibiotic administration to enable the concurrent or serial administration of multiple antimicrobial solutions at or above therapeutic targets such that multiple pathogens may be successfully treated within an acceptable clinical timeframe. For example, tobramycin may be administered once or twice daily for 2 hours, while vancomycin may be administered for the remainder of the day to maximize total daily exposure.

Generally, therapeutic drugs are developed with the goal of safely delivering the minimum effective dose to the patient for the treatment of a specific condition, symptom or set of symptoms. The clinical challenge of treating localized musculoskeletal infections such as or BII or FRI is that the specific pathogen(s) involved may not be known or may be polymicrobial (known or unknown) and are very likely biofilm forming and/or antibiotic resistant. Highly effective antibiotic eradication of localized infection in the presence of organisms which are antibiotic resistant and/or which have formed mature biofilm has not been achieved to date. This is due in large part to the direct conflict between the extremely high doses and concentrations of antibiotics required for eradication of such organisms and maintaining patient safety and to previously unknown doses, concentrations and combinations of antibiotics capable of biofilm eradication.

It should be appreciated that doses and concentrations of antibiotics and methods described herein may be described in the context of a particular infection, yet other infections or conditions are considered as well. For example, where a method is described as useful in periprosthetic joint infection, the method may be used in breast implant infection, another infected wound such as a traumatic wound, a diabetic ulcer, osteomyelitis, surgical site infection, etc. The antibiotics and methods described herein may be configured for use in any of a variety of localized infections and treatment sites beyond those specifically called out herein.

The treatment methods described herein may be performed manually (e.g., instillation of antibiotics by syringe and removal of fluids via a gravity drain), semi-automatically (e.g., instillation by syringe pump and aspiration by an active drainage device) or automatically (e.g., via a purpose-built irrigation delivery system). The irrigation delivery system may control irrigation (e.g., therapeutic antibiotic delivery and removal) of multiple antibiotics according to a set protocol including one or multiple sequential phases for delivery at one or multiple times and durations per day.

Also provided herein is a treatment delivery system and/or irrigation device for treating a local infection in a biological tissue of a human patient in need thereof. The treatment delivery system and/or irrigation device may be configured to locally administer a daily dose of vancomycin to the biological tissue that exceeds 3,500 mg and/or administer a daily dose of tobramycin that exceeds 150 mg.

In some embodiments, the treatment delivery system and/or irrigation device may further include an aspirating component and/or a flushing component. The aspirating component may be configured to apply suction to or around the biological tissue to remove any remaining antibiotic in the infection site. For example, the aspirating component may be configured to apply vacuum of at least-50 mmHg to the biological tissue for about 30 minutes. The flushing component may be configured to deliver a flushing fluid to the biological tissue to rinse/flush the infection site of any remaining antibiotic.

Further disclosed herein is a kit for treating a local infection in a biological tissue of a human patient. The kit may include at least one antibiotic, a treatment delivery system and/or irrigation device configured to locally irrigate (instill and aspirate) a series of doses of the at least one antibiotic, and instructions for administering a daily dose of the at least one antibiotic at a concentration that exceeds the MBEC level for the at least one antibiotic for the local infection. It is further contemplated that the instructions may include steps for administering the at least one antibiotic according to any of the methods disclosed herein. In some examples, the kit may include two or more antibiotics, a treatment delivery system and/or irrigation device, and instructions for administering the two or more antibiotics, where at least one of the antibiotics is at a concentration that exceeds the MBEC level for the at least one antibiotic for the local infection. The kit may include three or more antibiotics, a treatment delivery system and/or irrigation device, and instructions for administering the two or more antibiotics, where at least one of the antibiotics is at a concentration that exceeds the MBEC level for the at least one antibiotic for the local infection.

In another embodiment, the kit may include a first antibiotic combination of two or more antibiotics, a second antibiotic combination of two or more antibiotics, a treatment delivery system and/or irrigation device, and instructions for administering the first antibiotic combination and/or second antibiotic combination, wherein at least one of the antibiotics in the first or second antibiotic combination is at a concentration that exceeds the MBEC level for the at least one antibiotic for the local infection. In some examples, the treatment delivery system and/or irrigation device is configured to locally administer, via irrigation, a daily dose of vancomycin or daptomycin to the biological tissue that exceeds 3,500 mg and/or locally administer, via irrigation, a daily dose of tobramycin that exceeds 150 mg.

In some examples, the one or more, two or more, or three or more antibiotics may be selected from vancomycin, daptomycin, tobramycin, and/or any antibiotic known in the art. In some examples, the antibiotics may be provided in a vial or other container in an undiluted form. The end user may then combine the one or more antibiotics with saline or other biocompatible fluid in one or more fluid reservoirs at the desired concentration once the antibiotic(s) are ready to administer. In other examples, the antibiotics may be provided in saline and stored in fluid reservoirs, such as a bag. In various embodiments, the kit may include the one or more antibiotics and any combination of the treatment delivery system, irrigation device, fluid reservoirs, any other components needed for the treatment delivery system or irrigation device to function properly, and instructions for administering the antibiotic(s). In some instances, the kit may be provided separately to the end user such that separate elements of the kit may arrive to the end user at different times and then may be assembled by the end user prior to administration to a patient.

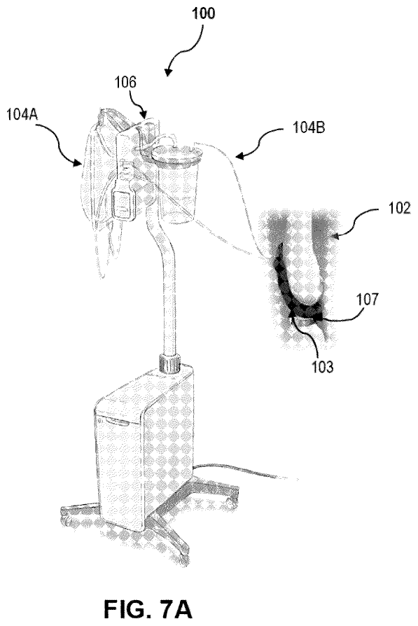

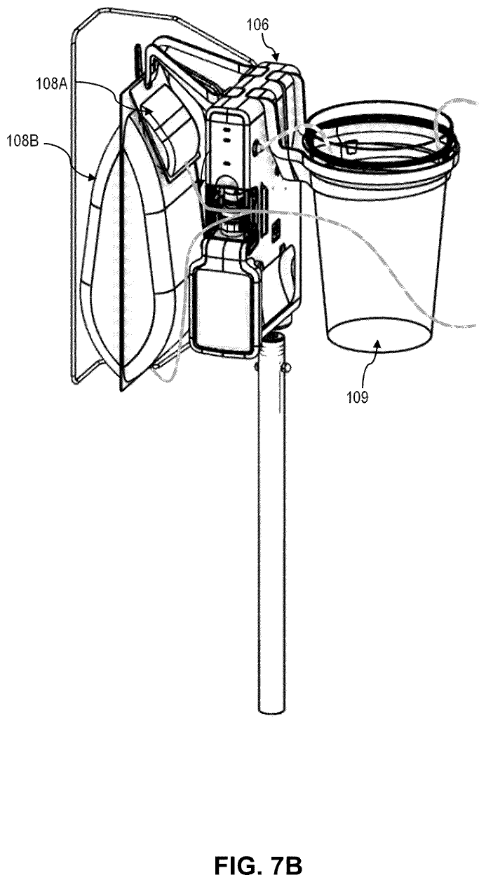

FIGS. 7A-7B show an example of a treatment delivery system 100 and irrigation device 107 that can provide controlled delivery of the one or more antibiotics into a treatment site 102 (e.g., infection in a periprosthetic space, bone segment, or wound area). All or portions of the treatment delivery system 100 and the irrigation device 107 can be included in the kit. In general, the treatment delivery system 100 can include a (portable) disposable device for enabling adjustment of a distance between the treatment delivery system 100 and a treated site 102 of a patient, such as a patient suffering from an infection treatable with fluid irrigation. In the example illustrated in FIG. 7A, the treatment delivery system 100 provides irrigation to a treatment site 102 including an irrigable volume (e.g., joint space or bone segment). The system 100 can include a fluid delivery system 104A, a fluid collection system 104B, and a control system 106. The fluid delivery system 104A can include one or more fluid reservoirs 108A, B. In some examples, the one or more fluid reservoirs 108A,B can contain the one or more antibiotics provided by the kit. For example, fluid reservoir 108A can contain a first antibiotic such as tobramycin in a saline solution and fluid reservoir 108B can contain a second antibiotic such as vancomycin or daptomycin in a saline solution. The fluid collection system 104B can include one or more collection fluid canisters 109.

In some examples, the control system 106 can control fluid flow from the fluid reservoirs 108A,B towards the treatment site 102 and/or draw fluid away from the treatment site 102 toward the fluid collection system 104B. For example, the control system 106 can include one or more of a vacuum pressure source, pump, or any other means for moving fluid through the system 100. In some examples, one means for moving the fluid may be used for both instilling and aspirating fluid from the treatment site 102. In other examples, one means may be used for instillation and another means may be used for aspiration. The system can be configured to deliver the fluid transcutaneously to irrigation devices deployed within treated area (e.g., infected tissue and/or infected joints, such as hip, knee, shoulder, wrist, ankle). Although not shown, one or more other medical devices may be assisting and working in coordination and/or parallel with the treatment delivery system 100 to provide treatment for the patient (including the treatment site 102).

In an example, the irrigation device 107 can be configured to be fluidly coupled to the treatment delivery system 100 as shown in FIG. 7A. The treatment delivery system 100 can also be coupled to a syringe or another manual component for delivery and withdrawal of fluid from the treatment site. In some aspects, the irrigation device 107 may have a porous component 103. The porous component 103 of the irrigation device 107 may be configured to fill the space and/or opening of the treatment site and allow for fluid exiting the tubing to flow through the porous component 103 into the treatment site 102.

The treatment system 100 can be configured to fluidly couple with the irrigation device 107 that can provide controlled delivery of fluids (e.g., antibiotics, saline, local anesthetics) into the treatment site 102 (e.g., an infected articulating joint, such as knee or hip) during, for example, I&D, DAIR, Double DAIR, or another procedure as described elsewhere herein or other type of healing including wound and perioperative healing.

Within a context example, the treatment delivery system 100 can be used for delivering one or more antibiotics to infected joints (e.g., hip, knee, shoulder, wrist, ankle) or any type of biological tissue that can be treated using controlled fluid irrigation. Examples of treatments include periprosthetic joint infection treatment protocols, such as debridement, antibiotics and implant retention (DAIR) and exchange arthroplasty, irrigation of surgical incisions and traumatic wounds or irrigation for osteomyelitis and septic arthritis. Although the application may be described in the context of a particular treatment site (e.g., infected joints) and connection with that site, it should be appreciated that other treatment sites are considered and the way in which the system connects with those various sites may vary.

Again with respect to FIGS. 7A-7B, the fluid delivery system 104A includes two or more fluid reservoir(s) 108A, 108B.

Each of the fluid reservoirs 108A, 108B can have a particular volume and store a particular fluid type that is delivered during a respective treatment phase. The contents of the fluid reservoirs may be switched such that a first antibiotic may be stored in a first fluid reservoir 108B or a second fluid reservoir 108A and a second antibiotic may be stored in a second fluid reservoir 108A or a first fluid reservoir 108B. The fluid reservoirs may also contain combinations of antibiotics in some instances. The fluid reservoirs may be provided as part of the kit or may be provided separately and combined with the one or more antibiotics of the kit prior to use.

For example, the first fluid reservoir 108A can contain or store a first fluid that can include an antibiotic (e.g., a first antibiotic), such as tobramycin, or a first antibiotic combination. The first fluid can be delivered from first fluid reservoir 108A to the treatment site 102 according to a respective fluid delivery protocol. The fluid delivery protocol can be included within the instructions of a kit. The protocol can define a concentration of a first antibiotic, a volume of the first antibiotic fluid to be delivered, a duration of antibiotic fluid delivery, and pre-delivery vacuum that can be performed for a set vacuum time period (e.g., approximately 30 minutes) at a set pressure (e.g., −125 mmHg). The volume of first antibiotic fluid to be delivered to the treatment site 102 can be set in a range between 6 mL and 500 mL, such as, 50 mL. In some implementations, approximately 80 mg of tobramycin sulfate in 50 mL of 0.9% sodium chloride are delivered in about 30 to 60 seconds and allowed to soak for a total of 2 hours in a single 24-hour period. The delivery of the first fluid volume can be controlled with an accuracy of +approximately 5 mL to 10 mL. In some implementations, the delivery of first antibiotic fluid is followed by a soaking protocol to enable the treatment site 102 to soak the delivered fluid. The duration of the first antibiotic fluid soaking protocol can be between 1 and 3 hours, such as approximately 2 hours. The first antibiotic fluid can be removed from the treatment site 102, by the fluid collection system 104B, before a second fluid is delivered to the treatment site 102. The duration of the first antibiotic fluid removal from the treatment site 102 can be approximately 30 minutes.

The second fluid reservoir 108B can store a second fluid that can include an antibiotic (e.g., a second antibiotic), such as vancomycin or daptomycin, or a second antibiotic combination. The volume of second fluid to be delivered to the treatment site 102 over at time period can be set in a range between 500 mL and 1500 mL, such as 1200 mL. The total volume of the second antibiotic fluid delivered can vary depending on the length of the time period as well as the total time for first fluid delivery, soak, and removal. For example, in a 24-hour time period where the first fluid delivery, soak, and removal was 2 hours, the time period of delivery of the second fluid can be approximately 22 hours. The delivery of the second antibiotic fluid volume can be controlled with an accuracy of +approximately 5 mL. The delivery of second antibiotic fluid is followed by a soaking protocol to enable the treatment site 102 to soak the delivered fluid. The duration of the second antibiotic fluid soaking protocol can be between 15 and 45 minutes, such as approximately 30 minutes. The second fluid can be removed from the treatment site 102, by the fluid collection system 104B. The duration of the second fluid removal from the treatment site 102 can be approximately 30 minutes. In some implementations, multiple cycles of second antibiotic fluid delivery, soaking, and removal are repeated before the first fluid is subsequently delivered to the treatment site 102. For example, the treatment protocol can be repeated over multiple (e.g., 5, 6, 7) days and during each day, the first antibiotic fluid can be delivered once to soak the treatment area 102 and after the removal of the first fluid, multiple cycles of second antibiotic fluid delivery, soaking, and removal can be repeated to complete a 24 hour treatment protocol. On Day 1, for example, the treatment site 102 and system can be prepped and a vacuum within the system established such as by a pump. Following seal checks and confirmations within the system, the first fluid can be delivered and allowed to soak as described above. After removal of the first fluid, the second fluid can be delivered, allowed to soak, and subsequently removed a plurality of times (e.g., 20-23 times) the remainder of day 1. At the start of day 2 (e.g., 24 hours after initiation of treatment), the canister and/or one or more reservoir bags can be changed and seal confirmed. A pre-treatment vacuum cycle can commence prior to initiation of the first fluid delivery and soak. After removal of the first fluid, the second fluid can be delivered, allowed to soak, and subsequently removed a plurality of times (e.g., 20-23 times) the remainder of day 2. This daily protocol can be repeated for a period of up to about 7 days, up to about 10 days, up to about 14 days, or however long the treatment is desired.

The fluid collection system 104B can direct fluid from the treatment site 102 to a collection fluid canister 109. In some examples, the control system 106 can generate a vacuum in the collection fluid canister 109 to remove the fluid from the treatment site 102 into the collection fluid canister 109. For example, a pump can be activated by the control system 106 to create a vacuum within at least the collection fluid canister 109. In other examples, vacuum pressure sources, syringes, gravity, pumps, or any other mechanism for moving fluid in and out of the treatment site 102 are contemplated. The collection fluid canister 109 can include a single-use canister with a known volume (e.g., 2000 mL). The collection fluid canister 109 collects antibiotic solution or other fluids (e.g., wound exudate) accumulated during vacuum-induced drainage of fluid from the treatment site 102. The material of the canister 109 is preferably translucent or transparent in order for a user to assess the level of the contents within the canister 109 during use. The material of the canister 109 is also preferably capable of retaining a vacuum without deforming or restricting flow at maximum vacuum level.

### EXAMPLES

### Example 1: Serum Concentrations for Vancomycin and Tobramycin Administered by Irrigation

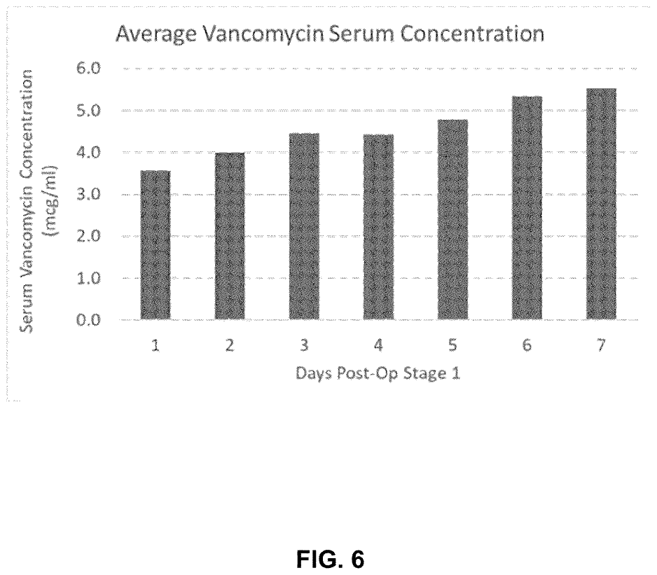

Irrigation of vancomycin and tobramycin within the manufacturer's recommended systemic dose for the treatment of PJI has been investigated clinically. A daily dose of 2.6 g vancomycin at a concentration of 2,500 mcg/mL and a daily dose of 80 mg of tobramycin at 1,600 mcg/mL were administered by irrigation to the infected joint for 7 days following stage one surgery of a two-stage exchange arthroplasty treatment. The serum concentration levels of vancomycin and tobramycin in this investigation were well below the safe serum concentration for each antibiotic. The serum concentrations of vancomycin over a 7-day period for 37 human subjects who received local irrigation of 50 cc at a concentration of 2,500 mcg/mL intermittently are shown in FIG. 6. Each dose was administered hourly, allowed to soak in the articulating joint (knee or hip) for 30 minutes and aspirated by applying vacuum of −125 mmHg to the joint for 30 minutes prior to repeating the hourly cycle 21.5 hours per day.

The peak serum concentrations of tobramycin over a 7-day period for 37 human clinical study subjects who received daily local irrigation of 50 cc at a concentration of 1,600 mcg/mL were minimal and below detectable limits in most subjects. Each dose was administered once daily, allowed to soak in the articulating joint (knee or hip) for 120 minutes and aspirated by applying vacuum of −125 mmHg to the joint for 30 minutes prior to repeating the hourly vancomycin irrigation cycle for the remaining 21.5 hours per day.

### Example 2: Tobramycin and Vancomycin on Pre-Formed Biofilms

The eradication of pre-formed biofilms from six clinically relevant strains using tobramycin and vancomycin was evaluated using a Modified BEST Assay™ Device. The BEST Assay™ devices and protocols were developed to perform high throughput testing of medical devices. The BEST Assay™ method quantitatively measures the microbial colonization on and around medical devices. The protocol presented herein was designed to evaluate the efficacy of a treatment of Tobramycin and Vancomycin against preformed biofilms, to determine its feasibility as a treatment for artificial joints that have been infected with established biofilms.

In particular, the study was performed to determine the anti-biofilm effect, against Pseudomonas aeruginosa, Escherichia coli, Staphylococcus aureus. Methicillin resistant Staphylococcus aureus (MRSA), Vancomycin Resistant Enterococcus faecalis (VRE), and Staphylococcus epidermidis of a treatment consisting of 2 hours of Tobramycin, followed by alternating 30 minutes of vancomycin/rest treatments either for 1 day or repeated for 7 days.

In a first test (code B in the tables below), tobramycin was administered at a concentration of 1,600 μg/mL in challenge media and was in contact with the biofilm for 2 hours, and then vancomycin was administered at a concentration of 2,500 μg/mL in challenge media and was in contact with the biofilm for 22 hours. In a second test (code C in the tables below), tobramycin was administered at a concentration of 1,600 μg/mL in challenge media and was in contact with the biofilm for 2 hours, and then vancomycin was administered to the biofilm at a concentration of 2,500 μg/mL in challenge media for 30 minute pulses over 22 hours. Table 1 provides a description of the samples and contact times. The challenge media included 20% TSB made with saline (0.9% NaCl) for S. aureus, S. epidermidis and E. faecalis and 10% TSB made with saline (0.9% NaCl) for E. coli and P. aeruginosa.

TABLE 1

| Code | Sample/Contact Time | |
|---|---|---|
| A | Challenge Media 2 h | Challenge Media 22 h |
| B | Tobramycin (1,600 μg/mL | Vancomycin (2,500 μg/mL |
| | in challenge media) 2 h | in challenge media) for 22 h |
| C | Tobramycin (1,600 μg/mL | Vancomycin (2,500 μg/mL |
| | in challenge media) 2 h | in challenge media) for 22 h |
| | | of 30-minute pulses |

The BEST Assay™ plates were assembled using hydroxyapatite coated Polyurethane/Acrylate coupons that were 8 mm×15 mm in size, which had been coated with hydroxyapatite. The BEST Assay™ plates were either standard or included three ports for each well to allow addition/removal of challenge media. Antimicrobial treatments were performed for 1 or 7 days.

### BEST Assay™ Device Setup:

This study was performed as two separate challenges (1 per time point). The one-day challenge was performed first, and the data from the 1-day challenge was used to modify the challenge set up before proceeding with the 7-day challenge.

### Subculture, Inoculum Preparation, and Biofilm Formation:

Using a cryogenic stock (at approximately −80° C.), a first subculture of the microorganisms listed above were streaked out on TSA plates. The plates were incubated at 37±2° C. for 20±4.0 hours and then stored, wrapped in parafilm, at ~4° C. until needed. From the first subculture plates, second subculture plates were streaked out as above and used within 32 hours. From the second subculture plates, single colonies were picked and used to inoculate ~10 mL of sterile TSB. The liquid cultures were incubated at 37±2° C. on a rotary shaker (110±10 rpm) for 20±2.0 hours.

The overnight culture was diluted in biofilm growth media (100% TSB). This was referred to as the inoculum, and was quantified by serial dilution and spot plating. The inoculated plates were incubated at 37±2° C. and counted after approximately 16-24 hours of incubation. Data was evaluated as total CFU recovered/device.

2 mL inoculum was added to the appropriate wells of the 24 well BEST Assay™ Device. The entire device was placed on a rotary shaker (110±10 rpm) and incubated at 37±2° C. for 24±2 hours.

### Challenge:

After the 24-hour incubation, the coupons were rinsed 1× with 2.5 mL/well saline for 30 seconds. The coupons were then transferred to either 2.5 mL/well tobramycin (concentration as listed Table 1) or 2.5 mL/well of challenge media, and incubatrf at 37±2° C. without shaking for 2 hours±10 minutes. Following the 2-hour incubation, coupons were rinsed 1× with 2.5 mL/well saline for 30 seconds. The coupons were moved to either 2.5 mL/well Vancomycin (Concentrations as listed in Table 1) or 2.5 mL of challenge media and the time was noted. For the standard BEST Assay™ plates, the plates were incubated for 22±1 hours at 37±2° C. without shaking.

For the modified BEST Assay™ plate, tubing connected to a vacuum pump, and two multichannel peristaltic pumps were attached to the BEST Assay™ Device. Vacuum and peristaltic pumps were controlled using Labview. Every 30 minutes the vacuum pump removed the media in each well of the device, followed by the peristaltic pumps immediately replacing the media with either the antibiotic solution or rinse solution. This occurred every 30 minutes for a total of 22±1 hour. For the challenges that were performed for more than 1 day, the steps above were repeated once per day (every 24±1 hour).

### Biofilm Recovery:

The coupon samples were rinsed in 2.5 mL sterile saline once. The coupons were placed in 2.5 mL of DE neutralizing broth.

The coupons were sonicated for 30 minutes. Following sonication, the coupons were transferred to 2.5 mL TSB, incubated for 24±2 hours at 37±2° C., and scored+/−for growth based on the development of turbidity. Following sonication, the recovery suspension was serially diluted and spot plated on TSA. The inoculated plates were incubated at 37±2° C. and counted after approximately 16-24 hours of incubation. Data was evaluated as total CFU recovered/device. Log₁₀ reduction was compared to control sample counts.

As the number of viable organisms recovered from the test coupons was expected to be low, a membrane filtration step was included for counting adhered organisms. The remaining recovery solution was filtered through a 0.45 μm filter membrane. Additional saline was added to ensure the recovery solution completely covered the membrane. Filters were placed on TSA plates, incubated at 37±2° C., and counted after 16-24 hours. All CFUs on the filter plate were counted.

### Results:

Tables 3a and 3b show a summary of the results for the 1 day administration with static tobramycin and vancomycin (code B according to Table 1) and pulsing vancomycin (code C according to Table 1) as compared to control (code A). Tables 4a and 4b show a summary of the comparison of code C to code B for the 1 day administration. The results show that the pulsing of the vancomycin results in a similar log reduction as compared to the static vancomycin administration, as both administration methods result in a Log₁₀ reduction, compared to the growth control, of greater than 4.0 for most of the biofilms.

TABLE 3a

| | | | Biofilm Recovery | | | | | |
|---|---|---|---|---|---|---|---|---|
| | | | Pseudomonas | | Escherichia | | Staphylococcus | |
| | | | aeruginosa | | coli | | aureus (MRSA) | |
| | | | LogR | % Kill | LogR | % Kill | LogR | % Kill |
| A | Vs. | B | 1.53 | 97.05% | 4.60 | 100.00% | 6.38 | 100.00% |
| | | C | 1.87 | 98.64% | 5.86 | 100.00% | 4.99 | 100.00% |

TABLE 3b

| | | | Biofilm Recovery | | | | | |
|---|---|---|---|---|---|---|---|---|
| | | | Staphylococcus | | Enterococcus | | Staphylococcus | |
| | | | aureus | | faecalis | | epidermidis | |
| | | | LogR | % Kill | LogR | % Kill | LogR | % Kill |
| A | Vs. | B | 5.91 | 100.00% | 4.86 | 100.00% | 6.50 | 100.00% |
| | | C | 4.93 | 100.00% | 5.96 | 100.00% | 4.72 | 100.00% |

TABLE 4a

| | | | Biofilm Recovery | | | | | |
|---|---|---|---|---|---|---|---|---|
| | | | Pseudomonas | | Escherichia | | Staphylococcus | |
| | | | aeruginosa | | coli | | aureus (MRSA) | |
| | | | LogR | % Kill | LogR | % Kill | LogR | % Kill |
| B | Vs. | C | 0.34 | −53.93% | 1.26 | 94.49% | −1.39 | −2377.53% |

TABLE 4b

| | | | Biofilm Recovery | | | | | |
|---|---|---|---|---|---|---|---|---|
| | | | Staphylococcus | | Enterococcus | | Staphylococcus | |
| | | | aureus | | faecalis | | epidermidis | |
| | | | LogR | % Kill | LogR | % Kill | LogR | % Kill |
| B | Vs. | C | −0.97 | −839.78% | −1.10 | 92.12% | −1.78 | −5982.46% |

Tables 5a and 5b show a summary of the results for the 7 day administration with static tobramycin and vancomycin (code B according to Table 1) and pulsing vancomycin (code C according to Table 1) as compared to control (code A). Tables 4a and 4b show a summary of the comparison of code C to code B for the 7 day administration. The results show that the pulsing of the vancomycin results in a similar log reduction as compared to the static vancomycin administration, as both administration methods result in a Log₁₀ reduction, compared to the growth control, of greater than 4.0 for most of the biofilms.

TABLE 5a

| | | | Biofilm Recovery | | | | | |
|---|---|---|---|---|---|---|---|---|
| | | | Pseudomonas | | Escherichia | | Staphylococcus | |
| | | | aeruginosa | | coli | | aureus (MRSA) | |
| | | | LogR | % Kill | LogR | % Kill | LogR | % Kill |
| A | Vs. | B | 6.44 | 100.00% | 7.13 | 100.00% | 5.90 | 100.00% |
| | | C | 3.18 | 99.93% | 5.72 | 100.00% | 5.45 | 100.00% |

TABLE 5b

| | | | Biofilm Recovery | | | | | |
|---|---|---|---|---|---|---|---|---|
| | | | Staphylococcus | | Enterococcus | | Staphylococcus | |
| | | | aureus | | faecalis | | epidermidis | |
| | | | LogR | % Kill | LogR | % Kill | LogR | % Kill |
| A | Vs. | B | 7.15 | 100.00% | 6.14 | 100.00% | 6.08 | 100.00% |
| | | C | 6.43 | 100.00% | 4.99 | 100.00% | 5.41 | 100.00% |

TABLE 6a

| | | | Biofilm Recovery | | | | | |
|---|---|---|---|---|---|---|---|---|
| | | | Pseudomonas | | Escherichia | | Staphylococcus | |
| | | | aeruginosa | | coli | | aureus (MRSA) | |
| | | | LogR | % Kill | LogR | % Kill | LogR | % Kill |
| B | Vs. | C | −3.26 | −181694.26% | −1.40 | −2431.13% | −0.45 | −184.71% |

TABLE 6b

| | | | Biofilm Recovery | | | | | |
|---|---|---|---|---|---|---|---|---|
| | | | Staphylococcus | | Enterococcus | | Staphylococcus | |
| | | | aureus | | faecalis | | epidermidis | |
| | | | LogR | % Kill | LogR | % Kill | LogR | % Kill |
| B | Vs. | C | −0.72 | −419.33% | −1.14 | −1288.29% | −0.68 | −373.99% |

### Example 3: Tobramycin and Vancomycin/Daptomycin to Eradicate Pre-Formed Biofilms

The eradication of pre-formed biofilms from six clinically relevant strains using tobramycin and vancomycin/daptomycin was evaluated using an MBEC Assay® Device. The MBEC Assay® device consists of a ninety-six (96) peg lid and a corresponding receiver plate with ninety-six (96) individual wells that have a maximum 200 μL working volume. Biofilm was established on the pegs under batch conditions (i.e., no flow of nutrients into or out of an individual well) with gentle mixing, resulting in the formation of 96 nearly identical biofilms. The ability of combinations of antimicrobial agents to remove pre-formed biofilms was determined by treating these pre-formed biofilms with the antimicrobial agents, and quantifying the cells remaining on the pegs. Log₁₀ reductions were calculated compared to a growth control composed of the challenge media without the added antimicrobial agent.

In particular, the study was performed to determine the anti-biofilm effect against Staphylococcus epidermidis, Staphylococcus aureus, Enterococcus faecalis, Escherichia coli and Pseudomonas aeruginosa, of treatments with varying concentrations of tobramycin and vancomycin/daptomycin. Varying concentrations (1,600 μg/mL to 40,000 μg/mL) of tobramycin were in contact with the biofilm for about 2 hours followed by varying concentrations of either vancomycin or daptomycin (2,500 μg/mL to 50,000 μg/mL) in contact with the biofilm for about 11 hours. Table 7 provides a description of the samples and contact times.

TABLE 7

| Code | Sample/Contact Time | |
|---|---|---|
| A | Tobramycin (1,600 μg/mL)/2 h | Vancomycin (2,500 μg/mL)/11 h |
| B | Tobramycin (14,400 μg/mL)/2 h | Vancomycin (2,500 μg/mL)/11 h |
| C | Tobramycin (27,200 μg/mL)/2 h | Vancomycin (2,500 μg/mL)/11 h |
| D | Tobramycin (40,000 μg/mL)/2 h | Vancomycin (2,500 μg/mL)/11 h |
| E | Tobramycin (1,600 μg/mL)/2 h | Vancomycin (18,333 μg/mL)/ |
| | | 11 h |
| F | Tobramycin (14,400 μg/mL)/2 h | Vancomycin (18,333 μg/mL)/ |
| | | 11 h |
| G | Tobramycin (27,200 μg/mL)/2 h | Vancomycin (18,333 μg/mL)/ |
| | | 11 h |
| H | Tobramycin (40,000 μg/mL)/2 h | Vancomycin (18,333 μg/mL)/ |
| | | 11 h |
| I | Tobramycin (1,600 μg/mL)/2 h | Vancomycin (34,167 μg/mL)/ |
| | | 11 h |
| J | Tobramycin (14,400 μg/mL)/2 h | Vancomycin (34,167 μg/mL)/ |
| | | 11 h |
| K | Tobramycin (27,200 μg/mL)/2 h | Vancomycin (34,167 μg/mL)/ |
| | | 11 h |
| L | Tobramycin (40,000 μg/mL)/2 h | Vancomycin (34,167 μg/mL)/ |
| | | 11 h |
| M | Tobramycin (1,600 μg/mL)/2 h | Vancomycin (50,000 μg/mL)/ |
| | | 11 h |
| N | Tobramycin (14,400 μg/mL)/2 h | Vancomycin (50,000 μg/mL)/ |
| | | 11 h |
| O | Tobramycin (27,200 μg/mL)/2 h | Vancomycin (50,000 μg/mL)/ |
| | | 11 h |
| P | Tobramycin (40,000 μg/mL)/2 h | Vancomycin (50,000 μg/mL)/ |
| | | 11 h |
| Q | Tobramycin (1,600 μg/mL)/2 h | Daptomycin (2,500 μg/mL)/11 h |
| R | Tobramycin (14,400 μg/mL)/2 h | Daptomycin (2,500 μg/mL)/11 h |
| S | Tobramycin (27,200 μg/mL)/2 h | Daptomycin (2,500 μg/mL)/11 h |
| T | Tobramycin (40,000 μg/mL)/2 h | Daptomycin (2.500 μg/mL)/11 h |
| U | Tobramycin (1,600 μg/mL)/2 h | Daptomycin (18,333 μg/mL)/ |
| | | 11 h |
| V | Tobramycin (14,400 μg/mL)/2 h | Daptomycin (18,333 μg/mL)/ |
| | | 11 h |
| W | Tobramycin (27,200 μg/mL)/2 h | Daptomycin (18,333 μg/mL)/ |
| | | 11 h |
| X | Tobramycin (40,000 μg/mL)/2 h | Daptomycin (18,333 μg/mL)/ |
| | | 11 h |
| Y | Tobramycin (1,600 μg/mL)/2 h | Daptomycin (34,167 μg/mL)/ |
| | | 11 h |
| Z | Tobramycin (14,400 μg/mL)/2 h | Daptomycin (34,167 μg/mL)/ |
| | | 11 h |
| AA | Tobramycin (27,200 μg/mL)/2 h | Daptomycin (34,167 μg/mL)/ |
| | | 11 h |
| AB | Tobramycin (40,000 μg/mL)/2 h | Daptomycin (34,167 μg/mL)/ |
| | | 11 h |
| AC | Tobramycin (1,600 μg/mL)/2 h | Daptomycin (50,000 μg/mL)/ |
| | | 11 h |
| AD | Tobramycin (14,400 μg/mL)/2 h | Daptomycin (50,000 μg/mL)/ |
| | | 11 h |
| AE | Tobramycin (27,200 μg/mL)/2 h | Daptomycin (50,000 μg/mL)/ |
| | | 11 h |
| AF | Tobramycin (40,000 μg/mL)/2 h | Daptomycin (50,000 μg/mL)/ |
| | | 11 h |

### Subculture, Inoculum Preparation, and Biofilm Formation:

Using a cryogenic stock (at approximately −80° C.), a first subculture of each of the microorganisms listed above was streaked on TSA plates. The plates were incubated at 37±2° C. for 20±4.0 hours and then stored, wrapped in parafilm, at ~4° C. until needed. From the first subculture plates, second subculture plates were streaked out as above and used within 32 hours. From the second subculture plates, single colonies were picked and used to inoculate 10-50 mL of sterile growth media. The liquid cultures were incubated at 37±2° C. on a rotary shaker (110±10 rpm) for 20±2.0 hours.

The overnight culture was diluted in biofilm growth media (100% TSB). This was referred to as the inoculum, and was quantified by serial dilution and spot plating. 150 μL inoculum was added to the appropriate wells of the 96 well plates, and the MBEC Assay® lids were placed on the plates. 150 μL sterile water was added to all of the remaining wells. The entire device was placed on a rotary shaker (110±10 rpm) and incubated at 37±2° C. for 24±2 hours.

### Challenge Plates:

A total of 6 MBEC Assay® Plates were used (1 uncoated and 2 HA coated per time point). Time points included a 13-hour and a 24-hour challenge.

Using sterile 96-well microtiter plates, the following was performed under sterile conditions to set up the MBEC Assay® first challenge plate and first tobramycin challenge. 200 μL sterile challenge media containing tobramycin at the concentrations stated in Table 7 was added to the appropriate wells of the 96 well plates. Some wells received 200 μL sterile challenge media that did not contain antibiotics. Media concentration (i.e., 10% or 20% TSB) matched what was being used on the rest of the plate. Some wells received 200 μL sterile challenge media containing 1,600 μg/mL tobramycin.

The MBEC Assay® lids, with adhered biofilms were transferred to new plates containing 200 μL/well saline for 30-60 seconds, and control pegs were broken off and placed in 200 μL of D/E recovery media and recovered. The lids were then transferred to the challenge plates for tobramycin in an unsealed bag with a damp paper towel to maintain humidity, and incubated at 37±2° C. for 2 h+15 min with no shaking.

An MBEC Assay® second challenge plate was set up for a first vancomycin or daptomycin challenge. 200 μL sterile challenge media, containing the desired antibiotic (vancomycin or daptomycin) at the concentrations stated in Table 7, was added to the appropriate wells of 96 well plates. Some wells received 200 μL sterile challenge media that did not contain antibiotics. Media concentration (i.e., 10% or 20% TSB) matched what was being used on the rest of the plate. Some wells received 200 μL sterile challenge media containing 2,500 μg/mL vancomycin or 2,500 μg/mL daptomycin.

The MBEC Assay® lids, with adhered biofilms were transferred to new plates containing 200 μL/well saline for 30-60 seconds. The lids were then transferred to the vancomycin or daptomycin challenge plates in an unsealed bag with a damp paper towel to maintain humidity, and incubated at 37±2° C. for 11±0.5 hours with no shaking. Following this incubation, the plates that were challenged for 13 hours were recovered while the plates that were to be challenged for 24-hours continued with further incubation. Following the 11-hour incubation, the MBEC Assay® lids with adhered biofilms were transferred to new plates containing 200 μL/well saline for 30-60 seconds. The MBEC Assay® lids with adhered biofilms were transferred to new plates containing 200 μL/well sterile challenge media without antibiotics in an unsealed bag with a damp paper towel to maintain humidity, and incubated at 37±2° C. for 11±2 hours with no shaking. Following this incubation, the plates that were challenged for 24 hours, were recovered.

### Biofilm Log₁₀ Reduction:

After desired challenge times, the quantity of biofilm remaining on the pegs was determined. MBEC Assay® lids were rinsed 3×30 seconds in sterile saline by transferring each lid to 3 sequential 96 well base plates containing 200 μL saline. The MBEC Assay® lids were transferred to recovery plates containing 200 μL per well of recovery media. The devices were sonicated for 30±5 min. Following sonication, the resulting CFU recovered were quantified by serially diluting and spot plating.

Plates were incubated at 37±2° C. and counted after 16-24 hours of incubation after which the plates were incubated for approximately 20 more hours and counted again. Data was evaluated as Log₁₀ CFU/peg and the log reduction compared to the growth control was calculated. 100 μL of 100% TSB was added to each well of the recovery plate and incubated for approximately 15 hours for the 13-hour time point and 24±2 hours for the 24-hour time point at 37±2° C. Following the incubation, 10 μL from each well was spotted onto a TSA plate and incubated for 16-24 hours at 37±2° C. and scored+/−for growth.

### Results:

Table 8a shows a summary of the Log R and % kill results for varying concentrations of tobramycin and vancomycin against Pseudomonas aeruginosa. Table 8b shows a summary of the Log R and % kill results for varying concentrations of tobramycin and daptomycin against Pseudomonas aeruginosa.

TABLE 8a

| | | | | Pseudomonas aeruginosa | | | | | | | |
|---|---|---|---|---|---|---|---|---|---|---|---|
| | | | | mg/mL Tobramycin | | | | | | | |
| | | | mg/mL | 1.600 | | 14.400 | | 27.200 | | 40.000 | |
| | | | Vancomycin | LogR | % Kill | LogR | % Kill | LogR | % Kill | LogR | % Kill |
| 13 | GC | Vs. | 2.500 | 4.16 | 99.99% | 4.16 | 99.99% | 6.24 | 100.00% | 4.33 | 100.00% |
| hours | | | 18.333 | 4.09 | 99.99% | 6.24 | 100.00% | 6.24 | 100.00% | 4.23 | 99.99% |
| | | | 34.167 | 4.45 | 100.00% | 6.24 | 100.00% | 4.92 | 100.00% | 6.24 | 100.00% |
| | | | 50.000 | 6.24 | 100.00% | 6.24 | 100.00% | 6.24 | 100.00% | 6.24 | 100.00% |
| 24 | GC | Vs. | 2.500 | 2.27 | 99.46% | 2.12 | 99.24% | 3.36 | 99.96% | 4.36 | 100.00% |
| hours | | | 18.333 | 3.46 | 99.97% | 3.76 | 99.98% | 4.06 | 99.99% | 6.27 | 100.00% |
| | | | 34.167 | 3.69 | 99.98% | 6.27 | 100.00% | 4.65 | 100.00% | 6.27 | 100.00% |
| | | | 50.000 | 6.27 | 100.00% | 6.27 | 100.00% | 6.27 | 100.00% | 6.27 | 100.00% |

TABLE 8b

| | | | | Pseudomonas aeruginosa | | | | | | | |
|---|---|---|---|---|---|---|---|---|---|---|---|
| | | | | mg/mL Tobramycin | | | | | | | |
| | | | mg/mL | 1.600 | | 14.400 | | 27.200 | | 40.000 | |
| | | | Daptomycin | LogR | % Kill | LogR | % Kill | LogR | % Kill | LogR | % Kill |
| 13 | GC | Vs. | 2.500 | 2.86 | 99.86% | 3.46 | 99.97% | 6.24 | 100.00% | 4.23 | 99.99% |
| hours | | | 18.333 | 3.94 | 99.99% | 6.24 | 100.00% | 4.16 | 99.99% | 4.63 | 100.00% |
| | | | 34.167 | 4.45 | 100.00% | 6.24 | 100.00% | 6.24 | 100.00% | 6.24 | 100.00% |
| | | | 50.000 | 4.45 | 100.00% | 6.24 | 100.00% | 4.45 | 100.00% | 3.73 | 99.98% |
| 24 | GC | Vs. | 2.500 | 0.27 | 45.96% | 6.27 | 100.00% | 6.27 | 100.00% | 6.27 | 100.00% |
| hours | | | 18.333 | 6.27 | 100.00% | 6.27 | 100.00% | 6.27 | 100.00% | 6.27 | 100.00% |
| | | | 34.167 | 6.27 | 100.00% | 6.27 | 100.00% | 6.27 | 100.00% | 6.27 | 100.00% |
| | | | 50.000 | 6.27 | 100.00% | 6.27 | 100.00% | 6.27 | 100.00% | 1.36 | 95.68% |

Table 9a shows a summary of the Log R and % kill results for varying concentrations of tobramycin and vancomycin against Escherichia coli. Table 9b shows a summary of the Log R and % kill results for varying concentrations of tobramycin and daptomycin against Escherichia coli.

TABLE 9a

| | | | | Escherichia coli | | | | | | | |
|---|---|---|---|---|---|---|---|---|---|---|---|
| | | | | mg/mL Tobramycin | | | | | | | |
| | | | mg/mL | 1.600 | | 14.400 | | 27.200 | | 40.000 | |
| | | | Vancomycin | LogR | % Kill | LogR | % Kill | LogR | % Kill | LogR | % Kill |
| 13 | GC | Vs. | 2.500 | 2.69 | 99.80% | 6.47 | 100.00% | 2.84 | 99.86% | 2.82 | 99.85% |
| hours | | | 18.333 | 4.16 | 99.99% | 4.26 | 99.99% | 6.47 | 100.00% | 4.26 | 99.99% |
| | | | 34.167 | 4.68 | 100.00% | 6.47 | 100.00% | 4.16 | 99.99% | 4.21 | 99.99% |
| | | | 50.000 | 6.47 | 100.00% | 6.47 | 100.00% | 6.47 | 100.00% | 3.86 | 99.99% |
| 24 | GC | Vs. | 2.500 | 6.63 | 100.00% | 4.84 | 100.00% | 6.63 | 100.00% | 6.63 | 100.00% |
| hours | | | 18.333 | 6.63 | 100.00% | 6.63 | 100.00% | 6.63 | 100.00% | 6.63 | 100.00% |
| | | | 34.167 | 6.63 | 100.00% | 6.63 | 100.00% | 6.63 | 100.00% | 6.63 | 100.00% |
| | | | 50.000 | 6.63 | 100.00% | 6.63 | 100.00% | 6.63 | 100.00% | 6.63 | 100.00% |

TABLE 9b

| | | | | Escherichia coli | | | | | | | |
|---|---|---|---|---|---|---|---|---|---|---|---|
| | | | | mg/mL Tobramycin | | | | | | | |
| | | | mg/mL | 1.600 | | 14.400 | | 27.200 | | 40.000 | |
| | | | Daptomycin | LogR | % Kill | LogR | % Kill | LogR | % Kill | LogR | % Kill |
| 13 | GC | Vs. | 2.500 | 4.12 | 99.99% | 3.09 | 99.92% | 3.39 | 99.96% | 4.39 | 100.00% |
| hours | | | 18.333 | 3.86 | 99.99% | 4.05 | 99.99% | 6.47 | 100.00% | 5.15 | 100.00% |
| | | | 34.167 | 4.16 | 99.99% | 4.86 | 100.00% | 4.16 | 99.99% | 6.47 | 100.00% |
| | | | 50.000 | 3.39 | 99.96% | 3.56 | 99.97% | 4.46 | 100.00% | 3.32 | 99.95% |
| 24 | GC | Vs. | 2.500 | 2.21 | 99.39% | 6.63 | 100.00% | 6.63 | 100.00% | 6.63 | 100.00% |
| hours | | | 18.333 | 6.63 | 100.00% | 6.63 | 100.00% | 6.63 | 100.00% | 6.63 | 100.00% |
| | | | 34.167 | 6.63 | 100.00% | 6.63 | 100.00% | 6.63 | 100.00% | 6.63 | 100.00% |
| | | | 50.000 | 6.63 | 100.00% | 2.85 | 99.86% | 6.63 | 100.00% | 6.63 | 100.00% |

Table 10a shows a summary of the Log R and % kill results for varying concentrations of tobramycin and vancomycin against Staphylococcus epidermidis. Table 10b shows a summary of the Log R and % kill results for varying concentrations of tobramycin and daptomycin against Staphylococcus epidermidis.

TABLE 10a

| | | | | Staphylococcus epidermidis | | | | | | | |
|---|---|---|---|---|---|---|---|---|---|---|---|
| | | | | mg/mL Tobramycin | | | | | | | |
| | | | mg/mL | 1.600 | | 14.400 | | 27.200 | | 40.000 | |
| | | | Vancomycin | LogR | % Kill | LogR | % Kill | LogR | % Kill | LogR | % Kill |
| 13 | GC | Vs. | 2.500 | 3.64 | 99.98% | 5.94 | 100.00% | 5.94 | 100.00% | 3.04 | 99.91% |
| hours | | | 18.333 | 5.94 | 100.00% | 5.94 | 100.00% | 2.64 | 99.77% | 5.94 | 100.00% |
| | | | 34.167 | 5.94 | 100.00% | 2.64 | 99.77% | 2.64 | 99.77% | 5.94 | 100.00% |
| | | | 50.000 | 1.94 | 98.86% | 1.94 | 98.86% | 2.16 | 99.31% | 2.16 | 99.31% |
| 24 | GC | Vs. | 2.500 | 4.14 | 99.99% | 6.48 | 100.00% | 3.34 | 99.95% | 6.48 | 100.00% |
| hours | | | 18.333 | 2.88 | 99.87% | 6.48 | 100.00% | 6.48 | 100.00% | 5.16 | 100.00% |
| | | | 34.167 | 2.82 | 99.85% | 4.57 | 100.00% | 4.87 | 100.00% | 4.70 | 100.00% |
| | | | 50.000 | 2.34 | 99.54% | 2.70 | 99.80% | 3.84 | 99.99% | 3.95 | 99.99% |

TABLE 10b

| | | | | Staphylococcus epidermidis | | | | | | | |
|---|---|---|---|---|---|---|---|---|---|---|---|
| | | | | mg/mL Tobramycin | | | | | | | |
| | | | mg/mL | 1.600 | | 14.400 | | 27.200 | | 40.000 | |
| | | | Daptomycin | LogR | % Kill | LogR | % Kill | LogR | % Kill | LogR | % Kill |
| 13 | GC | Vs. | 2.500 | 5.94 | 100.00% | 5.94 | 100.00% | 5.94 | 100.00% | 4.62 | 100.00% |
| hours | | | 18.333 | 5.94 | 100.00% | 5.94 | 100.00% | 5.94 | 100.00% | 5.94 | 100.00% |
| | | | 34.167 | 5.94 | 100.00% | 5.94 | 100.00% | 5.94 | 100.00% | 5.94 | 100.00% |
| | | | 50.000 | 5.94 | 100.00% | 5.94 | 100.00% | 5.94 | 100.00% | 5.94 | 100.00% |
| 24 | GC | Vs. | 2.500 | 6.48 | 100.00% | 6.48 | 100.00% | 6.48 | 100.00% | 4.70 | 100.00% |
| hours | | | 18.333 | 6.48 | 100.00% | 4.57 | 100.00% | 6.48 | 100.00% | 4.70 | 100.00% |
| | | | 34.167 | 5.16 | 100.00% | 6.48 | 100.00% | 6.48 | 100.00% | 4.87 | 100.00% |
| | | | 50.000 | 6.48 | 100.00% | 6.48 | 100.00% | 6.48 | 100.00% | 6.48 | 100.00% |

Table 11a shows a summary of the Log R and % kill results for varying concentrations of tobramycin and vancomycin against Enterococcus faecalis. Table 11b shows a summary of the Log R and % kill results for varying concentrations of tobramycin and daptomycin against Enterococcus faecalis.

TABLE 11a

| | | | | Enterococcus faecalis | | | | | | | |
|---|---|---|---|---|---|---|---|---|---|---|---|
| | | | | mg/mL Tobramycin | | | | | | | |
| | | | mg/mL | 1.600 | | 14.400 | | 27.200 | | 40.000 | |
| | | | Vancomycin | LogR | % Kill | LogR | % Kill | LogR | % Kill | LogR | % Kill |
| 13 | GC | Vs. | 2.500 | 2.05 | 99.11% | 2.42 | 99.62% | 2.20 | 99.37% | 2.28 | 99.48% |
| hours | | | 18.333 | 1.67 | 97.84% | 1.75 | 98.22% | 1.78 | 98.35% | 1.90 | 98.73% |
| | | | 34.167 | 1.27 | 94.67% | 1.90 | 98.73% | 1.78 | 98.35% | 1.82 | 98.48% |
| | | | 50.000 | 0.99 | 89.85% | 1.60 | 97.46% | 1.75 | 98.22% | 1.86 | 98.60% |
| 24 | GC | Vs. | 2.500 | 1.79 | 98.38% | 1.83 | 98.53% | 1.83 | 98.53% | 1.83 | 98.53% |
| hours | | | 18.333 | 1.69 | 97.94% | 1.69 | 97.94% | 1.58 | 97.35% | 1.83 | 98.53% |
| | | | 34.167 | 1.51 | 96.91% | 1.63 | 97.65% | 1.66 | 97.80% | 1.72 | 98.09% |
| | | | 50.000 | 1.72 | 98.09% | 1.79 | 98.38% | 1.88 | 98.68% | 1.79 | 98.38% |

TABLE 11b

| | | | | Enterococcus faecalis | | | | | | | |
|---|---|---|---|---|---|---|---|---|---|---|---|
| | | | | mg/mL Tobramycin | | | | | | | |
| | | | mg/mL | 1.600 | | 14.400 | | 27.200 | | 40.000 | |
| | | | Daptomycin | LogR | % Kill | LogR | % Kill | LogR | % Kill | LogR | % Kill |
| 13 | GC | Vs. | 2.500 | 6.20 | 100.00% | 6.20 | 100.00% | 6.20 | 100.00% | 6.20 | 100.00% |
| hours | | | 18.333 | 3.62 | 99.98% | 6.20 | 100.00% | 6.20 | 100.00% | 6.20 | 100.00% |
| | | | 34.167 | 6.20 | 100.00% | 6.20 | 100.00% | 6.20 | 100.00% | 6.20 | 100.00% |
| | | | 50.000 | 6.20 | 100.00% | 6.20 | 100.00% | 6.20 | 100.00% | 6.20 | 100.00% |
| 24 | GC | Vs. | 2.500 | 6.13 | 100.00% | 6.13 | 100.00% | 6.13 | 100.00% | 6.13 | 100.00% |
| hours | | | 18.333 | 6.13 | 100.00% | 6.13 | 100.00% | 6.13 | 100.00% | 3.79 | 99.98% |
| | | | 34.167 | 6.13 | 100.00% | 6.13 | 100.00% | 6.13 | 100.00% | 6.13 | 100.00% |
| | | | 50.000 | 6.13 | 100.00% | 6.13 | 100.00% | 6.13 | 100.00% | 6.13 | 100.00% |

Table 12a shows a summary of the Log R and % kill results for varying concentrations of tobramycin and vancomycin against Staphylococcus aureus. Table 12b shows a summary of the Log R and % kill results for varying concentrations of tobramycin and daptomycin against Staphylococcus aureus.

TABLE 12a

| | | | | Staphylococcus aureus | | | | | | | |
|---|---|---|---|---|---|---|---|---|---|---|---|
| | | | | mg/mL Tobramycin | | | | | | | |
| | | | mg/mL | 1.600 | | 14.400 | | 27.200 | | 40.000 | |
| | | | Vancomycin | LogR | % Kill | LogR | % Kill | LogR | % Kill | LogR | % Kill |
| 13 | GC | Vs. | 2.500 | 6.20 | 100.00% | 6.20 | 100.00% | 6.20 | 100.00% | 2.85 | 99.86% |
| hours | | | 18.333 | 6.20 | 100.00% | 6.20 | 100.00% | 6.20 | 100.00% | 6.20 | 100.00% |
| | | | 34.167 | 6.20 | 100.00% | 6.20 | 100.00% | 6.20 | 100.00% | 6.20 | 100.00% |
| | | | 50.000 | 6.20 | 100.00% | 6.20 | 100.00% | 6.20 | 100.00% | 6.20 | 100.00% |
| 24 | GC | Vs. | 2.500 | 5.57 | 100.00% | 5.57 | 100.00% | 5.57 | 100.00% | 5.57 | 100.00% |
| hours | | | 18.333 | 5.57 | 100.00% | 5.57 | 100.00% | 5.57 | 100.00% | 5.57 | 100.00% |
| | | | 34.167 | 5.57 | 100.00% | 5.57 | 100.00% | 5.57 | 100.00% | 5.57 | 100.00% |
| | | | 50.000 | 5.57 | 100.00% | 5.57 | 100.00% | 5.57 | 100.00% | 5.57 | 100.00% |

TABLE 12b

| | | | | Staphylococcus aureus | | | | | | | |
|---|---|---|---|---|---|---|---|---|---|---|---|
| | | | | mg/mL Tobramycin | | | | | | | |
| | | | mg/mL | 1.600 | | 14.400 | | 27.200 | | 40.000 | |
| | | | Daptomycin | LogR | % Kill | LogR | % Kill | LogR | % Kill | LogR | % Kill |
| 13 | GC | Vs. | 2.500 | 6.20 | 100.00% | 6.20 | 100.00% | 6.20 | 100.00% | 6.20 | 100.00% |
| hours | | | 18.333 | 6.20 | 100.00% | 6.20 | 100.00% | 6.20 | 100.00% | 6.20 | 100.00% |
| | | | 34.167 | 6.20 | 100.00% | 6.20 | 100.00% | 6.20 | 100.00% | 6.20 | 100.00% |
| | | | 50.000 | 6.20 | 100.00% | 6.20 | 100.00% | 6.20 | 100.00% | 6.20 | 100.00% |
| 24 | GC | Vs. | 2.500 | 5.57 | 100.00% | 5.57 | 100.00% | 5.57 | 100.00% | 5.57 | 100.00% |
| hours | | | 18.333 | 5.57 | 100.00% | 5.57 | 100.00% | 5.57 | 100.00% | 5.57 | 100.00% |
| | | | 34.167 | 5.57 | 100.00% | 5.57 | 100.00% | 5.57 | 100.00% | 5.57 | 100.00% |
| | | | 50.000 | 5.57 | 100.00% | 5.57 | 100.00% | 5.57 | 100.00% | 5.57 | 100.00% |

Table 13a shows a summary of the Log R and % kill results for varying concentrations of tobramycin and vancomycin against Staphylococcus aureus (MRSA). Table 13b shows a summary of the Log R and % kill results for varying concentrations of tobramycin and daptomycin against Staphylococcus aureus (MRSA).

TABLE 13a

| | | | | Staphylococcus aureus (MRSA) | | | | | | | |
|---|---|---|---|---|---|---|---|---|---|---|---|
| | | | | mg/mL Tobramycin | | | | | | | |
| | | | mg/mL | 1.600 | | 14.400 | | 27.200 | | 40.000 | |
| | | | Vancomycin | LogR | % Kill | LogR | % Kill | LogR | % Kill | LogR | % Kill |
| 13 | GC | Vs. | 2.500 | 1.11 | 92.20% | 1.31 | 95.13% | 1.53 | 97.08% | 1.41 | 96.10% |
| hours | | | 18.333 | 1.97 | 98.93% | 1.90 | 98.73% | 1.73 | 98.15% | 0.76 | 82.46% |
| | | | 34.167 | 6.31 | 100.00% | 1.71 | 98.05% | 2.01 | 99.03% | 1.71 | 98.05% |
| | | | 50.000 | 1.01 | 90.25% | 1.71 | 98.05% | 1.31 | 95.13% | 2.01 | 99.03% |
| 24 | GC | Vs. | 2.500 | 0.70 | 80.00% | 0.90 | 87.50% | 0.90 | 87.50% | 0.49 | 67.50% |
| hours | | | 18.333 | 1.37 | 95.75% | 1.40 | 96.00% | 1.28 | 94.75% | 1.05 | 91.00% |
| | | | 34.167 | 1.90 | 98.75% | 1.65 | 97.75% | 1.76 | 98.25% | 1.40 | 96.00% |
| | | | 50.000 | 1.35 | 95.50% | 1.46 | 96.50% | 2.12 | 99.25% | 1.22 | 94.00% |

TABLE 13b

| | | | | Staphylococcus aureus (MRSA) | | | | | | | |
|---|---|---|---|---|---|---|---|---|---|---|---|
| | | | | mg/mL Tobramycin | | | | | | | |
| | | | mg/mL | 1.600 | | 14.400 | | 27.200 | | 40.000 | |
| | | | Daptomycin | LogR | % Kill | LogR | % Kill | LogR | % Kill | LogR | % Kill |
| 13 | GC | Vs. | 2.500 | 4.99 | 100.00% | 4.23 | 99.99% | 3.86 | 99.99% | 3.93 | 99.99% |
| hours | | | 18.333 | 6.31 | 100.00% | 4.01 | 99.99% | 4.01 | 99.99% | 6.31 | 100.00% |
| | | | 34.167 | 6.31 | 100.00% | 3.71 | 99.98% | 4.01 | 99.99% | 3.71 | 99.98% |
| | | | 50.000 | 6.31 | 100.00% | 6.31 | 100.00% | 2.83 | 99.85% | 2.83 | 99.85% |
| 24 | GC | Vs. | 2.500 | 5.90 | 100.00% | 5.90 | 100.00% | 5.90 | 100.00% | 5.90 | 100.00% |
| hours | | | 18.333 | 5.90 | 100.00% | 5.90 | 100.00% | 5.90 | 100.00% | 5.90 | 100.00% |
| | | | 34.167 | 5.90 | 100.00% | 5.90 | 100.00% | 5.90 | 100.00% | 5.90 | 100.00% |
| | | | 50.000 | 5.90 | 100.00% | 4.58 | 100.00% | 5.90 | 100.00% | 4.58 | 100.00% |

### Definitions

In various implementations, description is made with reference to the figures. However, certain implementations may be practiced without one or more of these specific details, or in combination with other known methods and configurations. In the description, numerous specific details are set forth, such as specific doses, fixed dose combinations and concentrations, in order to provide a thorough understanding of the invention. In other instances, specific doses, fixed dose combinations and concentrations have not been described in particular detail in order to not unnecessarily obscure the description. Reference throughout this specification to “dose,” “fixed dose ratio,” “concentration” or the like, means that a particular method, or characteristic described is included in at least one novel method of treatment. Thus, the appearance of the phrase “one method of treatment,” or the like, in various places throughout this specification is not necessarily referring to the same method of treatment. Furthermore, the particular antibiotics, doses, fixed dose combinations, ratios and concentrations may be combined in any suitable manner in one or more novel method of treatment.

Where a range of values is provided, each value between the upper and lower ends of the range are specifically contemplated and described herein.

As used herein, the term “about” means a range of values including the specified value, which a person of ordinary skill in the art would consider reasonably similar to the specified value. In aspects, about means within a standard deviation using measurements generally acceptable in the art. In aspects, about means a range extending to +/−10% of the specified value and may include the specified value. For example, the endpoint may be within 10%, 8%, 5%, 3%, 2%, or 1% of the listed value. Further, for the sake of convenience and brevity, a numerical range of “about 50 mg/mL to about 80 mg/mL” should also be understood to provide support for the range of “50 mg/mL to 80 mg/mL” The endpoint may also be based on the variability allowed by an appropriate regulatory body, such as the FDA, USP, etc.

As used herein, the term “substantially” means a value a person of ordinary skill in the art would consider significantly more than a specified value. In some aspects, substantially means at least 25% more than the specified value, or at least 125% of the specified value.

As used herein, the terms “safe” and “recommended” means recommended by a medical authority, such as a manufacturer, the FDA or other similar regulatory body. In some examples, the maximum dose or serum level may be based on peer reviewed medical literature, for example Rybak et al., “Therapeutic Monitoring of Vancomycin in Adult Patients: A Consensus Review of the American Society of Health-System Pharmacists, the Infections Diseases Society of America, and the Society of Infectious Diseases Pharmacists” ASHP Therapeutic Position Statements, p. 685-700, 2009, or recommended by a professional medical organization or association. In additional examples, a maximum recommended daily systemic dose and/or a maximum safe serum concentration may be provided in the label or instructions for use of the particular antibiotic.

As used herein, the terms “maximum recommended daily dose”, “maximum recommended daily systemic dose” and “maximum recommended systemic daily dose” may be used interchangeably. In some examples, the systemic daily dose may be a daily dose administered by IV.

While this specification contains many specifics, these should not be construed as limitations on the scope of what is claimed or of what may be claimed, but rather as descriptions of features specific to particular methods. Certain methods that are described in this specification may also be implemented in combination in a single method. Conversely, various features that are described in the context of a single method may also be implemented individually, in multiple methods separately or in any suitable sub-combination of novel methods. Moreover, although methods may be described herein as including certain combinations of antibiotics, doses or concentrations and even initially claimed as such, one or more features from such a claimed combination may in some cases be excised from the combination, and the claimed combination may be directed to a sub-combination or a variation of a sub-combination. Similarly, while administration of more than one antibiotic sequentially is described in the specification in a particular order or for particular durations, this should not be understood as requiring that such methods be performed in the particular sequential order or for the particular durations, or that all elements of the sequence be performed, to achieve desirable results. Only a few examples and methods are disclosed. Variations, modifications and enhancements to the described methods and other methods may be made based on what is disclosed.

In the descriptions above and in the claims, phrases such as “at least one” or “one or more” may occur followed by one or a conjunctive list of elements, antibiotics, combinations, doses or concentrations. The term “and/or” may also occur in a list of two or more such elements. Unless otherwise implicitly or explicitly contradicted by the context in which it is used, such a phrase is intended to mean any of the listed elements individually or any of the recited elements in combination with any of the other recited elements. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on” above and in the claims is intended to mean, “based at least in part on,” such that an unrecited feature or element is also permissible.

As used herein, “comprises,” “comprising,” “containing,” and “having” and the like may have the meaning ascribed to them in U.S. Patent Law and may mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. In this specification when using an open-ended term, like “comprising” or “including,” it is understood that direct support should be afforded also to “consisting essentially of” language as well as “consisting of” language as if stated explicitly and vice versa.

As used herein, first, second, third, etc. are used to characterize and distinguish various antibiotics. Use of numerical terms may be used to distinguish one antibiotic from another antibiotic. Use of such numerical terms does not imply a sequence or order unless clearly indicated by the context. Such numerical references may be used interchangeably without departing from the teaching of the embodiments and variations herein.

The inventions disclosed herein may be packaged together in a single package of multiple antibiotics or in multiple packages of individual or combinations of antibiotics for use together, and such use may be administered concurrently, serially, cyclically or sequentially (in a particular sequence which may include repeat use of one or more individual or combination of antibiotics) per specific instructions.

All methods described herein may be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claims. No language in the specification should be construed as indicating any non-claimed element essential to the practice of the invention.

### Embodiments

Embodiment 1: A method of treating a localized infection of a human patient, the method comprising: locally administering a solution to the human patient in a 24-hour period a dose of at least one antimicrobial agent that substantially exceeds a maximum recommended daily systemic dose of the at least one antimicrobial agent; and during the 24-hour period providing a peak or trough serum concentration less than or equal to a maximum safe level for serum concentration of the at least one antimicrobial agent.

Embodiment 2: The method of embodiment 1, wherein the locally administering comprises irrigating the localized infection with the at least one antimicrobial agent one or more times.

Embodiment 3: The method of embodiment 2, wherein the irrigating the localized infection comprises soaking the local site of infection with the at least one antimicrobial agent and subsequently aspirating the at least one antimicrobial agent from the local site of infection.

Embodiment 4: The method of embodiment 3, wherein the soaking is for about 10 minutes to about 180 minutes.

Embodiment 5: The method of embodiment 4, wherein the soaking is for about 30 minutes to about 120 minutes.

Embodiment 6: The method of embodiment 3, wherein the irrigating is performed at least twice in the 24-hour period.

Embodiment 7: The method of embodiment 3, wherein the irrigating is performed at least 5 times in the 24-hour period.

Embodiment 8: The method of embodiment 3, wherein the irrigating is performed at least 10 times in the 24-hour period.

Embodiment 9: The method of embodiment 3, wherein the irrigating is performed at least 15 times in the 24-hour period.

Embodiment 10: The method of embodiment 3, wherein the irrigating is performed at least 20 times in the 24-hour period.

Embodiment 11: The method of embodiment 1, wherein the locally administered dose of the at least one antimicrobial agent in the 24-hour period exceeds the maximum recommended daily systemic dose of the at least one antimicrobial agent by at least 125%.

Embodiment 12: The method of embodiment 1, wherein the locally administered dose of the at least one antimicrobial agent in the 24-hour period exceeds the maximum recommended daily systemic dose of the at least one antimicrobial agent by at least 150%.

Embodiment 13: The method of embodiment 1, wherein the locally administered dose of the at least one antimicrobial agent in the 24-hour period exceeds the maximum recommended daily systemic dose of the at least one antimicrobial agent by at least 175%.

Embodiment 14: The method of embodiment 1, wherein the locally administered dose of the at least one antimicrobial agent in the 24-hour period exceeds the maximum recommended daily systemic dose of the at least one antimicrobial agent by at least 200%.

Embodiment 15: The method of embodiment 1, wherein the at least one antimicrobial agent is an antibiotic.

Embodiment 16: The method of embodiment 15, wherein the at least one antibiotic is an aminoglycoside.

Embodiment 17: The method of embodiment 15, wherein the at least one antibiotic is a glycopeptide.

Embodiment 18: The method of embodiment 17, wherein the at least one antibiotic is vancomycin.

Embodiment 19: The method of embodiment 18, wherein the locally administered dose of vancomycin is at least 3,000 mg in the 24-hour period.

Embodiment 20: The method of embodiment 18, wherein the locally administered dose of vancomycin is at least 6,000 mg in the 24-hour period.

Embodiment 21: The method of embodiment 18, wherein the locally administered dose of vancomycin is at least 12,000 mg in the 24-hour period.

Embodiment 22: The method of embodiment 18, wherein the locally administered dose of vancomycin is at least 24,000 mg in the 24-hour period.

Embodiment 23: The method of embodiment 18, wherein the locally administered daily dose of vancomycin is 3,000 to 50,000 mg in the 24-hour period, and the trough serum concentration of vancomycin is less than 30 mcg/mL.

Embodiment 24: The method of embodiment 23, wherein the trough serum concentration of vancomycin is less than 20 mcg/mL.

Embodiment 25: The method of embodiment 23, wherein the trough serum concentration of vancomycin is less than 10 mcg/mL.

Embodiment 26: The method of embodiment 18, wherein the maximum safe level for trough serum concentration of vancomycin is 25 mcg/mL.

Embodiment 27: The method of embodiment 18, wherein locally administering comprises irrigating the localized infection with the vancomycin two or more times in the 24-hour period.

Embodiment 28: The method of embodiment 27, wherein irrigating the localized infection comprises soaking the localized infection with the vancomycin and subsequently aspirating the vancomycin from the site of the localized infection.

Embodiment 29: The method of embodiment 18, wherein the localized infection comprises an unidentified pathogen.

Embodiment 30: The method of embodiment 18, wherein the localized infection is polymicrobial.

Embodiment 31: The method of embodiment 18, wherein the site of the localized infection is a topical wound.

Embodiment 32: The method of embodiment 31, wherein the topical wound is a diabetic ulcer.

Embodiment 33: The method of embodiment 18, wherein the site of the localized infection is a traumatic wound.

Embodiment 34: The method of embodiment 33, wherein the traumatic wound includes and implanted medical device.

Embodiment 35: The method of embodiment 18, wherein the site of the localized infection is a surgical incision.

Embodiment 36: The method of embodiment 18, wherein the site of the localized infection is a subcutaneous surgical wound.

Embodiment 37: The method of embodiment 36, wherein the subcutaneous surgical wound includes an implanted medical device.

Embodiment 38: The method of embodiment 37, wherein the implanted medical device is an orthopedic implant.

Embodiment 39: The method of embodiment 38, wherein the orthopedic implant is a joint replacement prosthesis.

Embodiment 40: The method of embodiment 16, wherein the aminoglycoside is tobramycin.

Embodiment 41: The method of embodiment 40, wherein the locally administered dose of tobramycin is at least 100 mg in the 24-hour period.

Embodiment 42: The method of embodiment 40, wherein the locally administered dose of tobramycin is at least 200 mg in the 24-hour period.

Embodiment 43: The method of embodiment 40, wherein the daily dose of tobramycin is at least 400 mg in the 24-hour period.

Embodiment 44: The method of embodiment 40, wherein the daily dose of tobramycin is at least 800 mg in the 24-hour period.

Embodiment 45: The method of embodiment 40, wherein the daily dose of tobramycin is 100 to 1,000 mg/day, and the peak serum concentration of tobramycin is less than 10 mcg/mL.

Embodiment 46: The method of embodiment 40, wherein the peak serum concentration of tobramycin is less than 9 mcg/mL.

Embodiment 47: The method of embodiment 40, wherein the peak serum concentration of tobramycin is less than 6 mcg/mL.

Embodiment 48: The method of embodiment 40, wherein the peak serum concentration of tobramycin is less than 3 mcg/mL.

Embodiment 49: The method of embodiment 40, wherein the peak serum concentration of tobramycin is less than a detectable limit of a clinical laboratory test for serum concentration of tobramycin.

Embodiment 50: The method of embodiment 1, wherein the at least one antibiotic comprises two antibiotics.

Embodiment 51: The method of embodiment 50, wherein the two antibiotics comprise vancomycin and tobramycin.

Embodiment 52: The method of embodiment 51, wherein the two antibiotics are sequentially administered.

Embodiment 53: The method of embodiment 52, wherein the vancomycin is locally administered hourly for 20 to 22 hours in the 24-hour period, wherein the vancomycin soaks for about 30 minutes and subsequently aspirated for about 30 minutes, and wherein the tobramycin is locally administered once in the 24-hour period for about 2 hours.

Embodiment 54: The method of embodiment 51, wherein the locally administered concentration of the vancomycin is 3,000 mcg/mL to 50,000 mcg/mL and the locally administered concentration of the tobramycin is 2,000 mcg/mL to 40,000 mcg/mL.

Embodiment 55: The method of embodiment 51, wherein the local administration is provided for 1 to 10 days.

Embodiment 56: The method of embodiment 51, wherein the vancomycin and tobramycin are administered at a fixed dose ratio of 6:1 to 150:1.

Embodiment 57: The method of embodiment 51, wherein the dose of vancomycin is at least 3,000 mg in the 24-hour period or the dose of tobramycin is at least 100 mg in the 24-hour period.

Embodiment 58: The method of embodiment 51, wherein the dose of vancomycin is at least 6,000 mg in the 24-hour period or the dose of tobramycin is at least 200 mg in the 24-hour period.

Embodiment 59: The method of embodiment 51, wherein the dose of vancomycin is at least 12,000 mg in the 24-hour period or the dose of tobramycin is at least 400 mg in the 24-hour period.

Embodiment 60: The method of embodiment 51, wherein the daily dose of vancomycin is 3,000 to 50,000 mg in the 24-hour period or the daily dose of tobramycin is 100-4,000 mg in the 24-hour period, and the trough serum concentration of vancomycin is less than 30 mcg/mL and the peak serum concentration of tobramycin is less than 10 mcg/mL.

Embodiment 61: A method of treating a localized infection of a human patient, the method comprising: locally administering to the human patient a locally administered concentration of at least one antimicrobial agent that exceeds a maximum recommended concentration of the at least one antimicrobial agent; and providing a peak or trough serum concentration less than a maximum safe serum concentration of the at least one antimicrobial agent.

Embodiment 62: The method of embodiment 61, wherein the locally administering comprises irrigating the localized infection by soaking the local infection with the at least one antimicrobial agent and aspirating the at least one antimicrobial agent after a period of time.

Embodiment 63: The method of embodiment 62, wherein the at least one antimicrobial agent is vancomycin.

Embodiment 64: The method of embodiment 62, wherein the concentration of vancomycin is at least 3,000 mcg/mL.

Embodiment 65: The method of embodiment 62, wherein the locally administered concentration of vancomycin is 3,000 to 50,000 mcg/mL, and the trough serum concentration is less than 30 mcg/mL.

Embodiment 66: The method of embodiment 62, wherein the at least one antimicrobial agent is tobramycin.

Embodiment 67: The method of embodiment 66, wherein the locally administered concentration of tobramycin is at least 100 mcg/mL.

Embodiment 68: The method of embodiment 66, wherein the locally administered concentration of tobramycin is 100 to 40,000 mcg/mL, and the peak serum concentration is less than 10 mcg/mL.

Embodiment 69: The method of embodiment 62, wherein the at least one antimicrobial agent comprises vancomycin and tobramycin.

Embodiment 70: The method of embodiment 69, wherein the locally administered concentration of vancomycin is at least 3,000 mcg/mL or the locally administered concentration of tobramycin is at least 100 mcg/mL.

Embodiment 71: The method of embodiment 69, wherein the locally administered concentration of vancomycin is 3,000 to 50,000 mcg/mL or the locally administered concentration of tobramycin is 100 to 40,000 mcg/mL, and the trough serum concentration of vancomycin is less than 30 mcg/mL and the peak serum concentration of tobramycin is less than 10 mcg/mL.

Embodiment 72: A method of treating a musculoskeletal infection of a human patient comprising: administering to the human patient at least one antimicrobial agent at a locally administered concentration that exceeds a minimum biofilm eradication concentration (MBEC) of the at least one antimicrobial agent; and providing a peak or trough serum concentration of the at least one antimicrobial agent less than a maximum safe serum concentration for the at least one antimicrobial agent.

Embodiment 73: The method of embodiment 72, wherein the MBEC comprises the concentration at which the at least one antimicrobial agent reduces the number of colony forming units (CFU's) of a specific microbe that has formed a biofilm for at least 24 hours by greater than 99.999% within a period of time over which the at least one antimicrobial agent is locally administered to the localized infection.

Embodiment 74: The method of embodiment 73, wherein the microbe is one or more of Staphylococcus aureus, methicillin resistant Staphylococcus aureus (MRSA), vancomycin resistant Enterococcus faecalis (VRE), Staphylococcus epidermidis, Enterococcus, Escherichia coli, and Pseudomonas aeruginosa.

Embodiment 75: The method of embodiment 72, wherein the at least one antimicrobial agent comprises a glycopeptide and an aminoglycoside.

Embodiment 76: The method of embodiment 75, wherein the glycopeptide comprises vancomycin and the aminoglycoside comprises tobramycin.

Embodiment 77: The method of embodiment 76, wherein the locally administered concentration of vancomycin is at least 3,000 mcg/mL and the locally administered concentration of tobramycin is at least 100 mcg/mL.

Embodiment 78: The method of embodiment 76, wherein the locally administered concentration of vancomycin is 3,000 to 50,0000 mcg/mL and the locally administered concentration of tobramycin is 100 to 40,000 mcg/mL, and the peak serum concentration of tobramycin is less than 10 mcg/mL and the trough serum concentration of vancomycin is less than 30 mcg/mL.

Embodiment 79: A method of treating a localized infection of a human patient, the method comprising: locally administering to the human patient, in a 24-hour period, a total dose of a first antibiotic that substantially exceeds a maximum recommended systemic daily dose of the first antibiotic; and providing a peak or trough serum concentration during the 24-hour period less than or equal to a maximum safe serum concentration of the first antibiotic.

Embodiment 80: A method of treating a localized infection of a human patient in need thereof, the method comprising: locally administering to the human patient, in a 24-hour period, a total dose of a first antibiotic that substantially exceeds a maximum recommended daily systemic dose of the first antibiotic and a total dose of a second antibiotic that substantially exceeds a maximum recommended daily dose of the second antibiotic; and providing a peak or trough serum concentration, during the 24-hour period, less than or equal to a maximum recommended safe serum concentration of the first antibiotic and the second antibiotic.

Embodiment 81: A method of treating a localized infection of a human patient, the method comprising: locally administering to the human patient, in a 24-hour period, a total dose of at least two antibiotics that substantially exceeds a maximum recommended daily systemic dose of each of the at least two antibiotics; and providing a peak or trough serum concentration, during the 24-hour period, less than or equal to a maximum safe serum concentration of each of the at least two antibiotics.

Embodiment 82: A method of treating a biological tissue of a human patient, the method comprising: locally administering to the human patient, in a 24-hour period, a total dose of at least one therapeutic agent that substantially exceeds a maximum recommended systemic daily dose of the at least one therapeutic agent; and providing a peak or trough serum concentration less than or equal to a maximum safe serum concentration of the at least one therapeutic agent.

Embodiment 83: A method of treating a localized infection in a biological tissue of a human patient, the method comprising: locally administering to the human patient, in a 24-hour period, a total dose of at least one antibiotic that exceeds 3,000 mg in the 24-hour period.

Embodiment 84: A method of treating a localized infection in a human patient, the method comprising: locally administering to the human patient, in a 24-hour period, a total dose of vancomycin that exceeds 3,000 mg/day.

Embodiment 85: The method of embodiment 84, wherein the total dose of vancomycin in the 24-hour period is at least 6,000 mg/day.

Embodiment 86: The method of embodiment 84, wherein the total dose of vancomycin in the 24-hour period is at least 12,000 mg/day.

Embodiment 87: The method of embodiment 84, wherein the locally administered total dose of vancomycin in the 24-hour period is at least 24,000 mg/day.

Embodiment 88: A method of treating a localized infection in a human patient, the method comprising: locally administering to the human patient, in a 24-hour period, a total dose of tobramycin that exceeds 100 mg in the 24-hour period.

Embodiment 89: The method of embodiment 88, wherein the dose of tobramycin is at least 200 mg in the 24-hour period.

Embodiment 90: The method of embodiment 88, wherein the dose of tobramycin is at least 400 mg in the 24-hour period.

Embodiment 91: The method of embodiment 88, wherein the dose of tobramycin is at least 800 mg in the 24-hour period.

Embodiment 92: A kit for treating a localized infection in a human patient, the kit comprising: at least one antibiotic; a treatment delivery system and/or irrigation device configured to locally irrigate the localized infection with at least one dose of the at least one antibiotic; and instructions for administering the at least one dose of the at least one antibiotic.

Embodiment 93: The kit of embodiment 92, wherein the at least one antibiotic is vancomycin.

Embodiment 94: The kit of embodiment 92, wherein the treatment delivery system and/or irrigation device is configured to locally administer in a 24-hour period a total dose of vancomycin that exceeds 3,000 mg.

Embodiment 95: The kit of embodiment 92, wherein the at least one antibiotic is tobramycin.

Embodiment 96: The kit of embodiment 92, wherein the treatment delivery system and/or irrigation device is configured to locally administer in a 24-hour period a total dose of tobramycin that exceeds 100 mg.

Embodiment 97: The kit of embodiment 92, wherein the kit comprises at least two antibiotics, wherein at least one of the antibiotics is at a concentration that exceeds a minimum biofilm eradication concentration (MBEC) of the at least one antibiotic.

Embodiment 98: The kit of embodiment 92, wherein the at least one antibiotic comprises vancomycin and tobramycin.

Embodiment 99: The kit of embodiment 97, wherein the at least two antibiotics comprises daptomycin and tobramycin.

Embodiment 100: The kit of embodiment 92, wherein the at least one antibiotic is provided in a fluid reservoir.

Embodiment 101: A method of treating a localized infection in a human patient, the method comprising: locally administering to the human patient two or more doses within a 24-hour period of at least one antibiotic at a concentration that exceeds a MBEC level for the at least one antibiotic for the local infection.

Embodiment 102: The method of embodiment 101, comprising: locally administering five or more doses within the 24-hour period of the at least one antibiotic at a concentration that exceeds the MBEC level for the at least one antibiotic for the local infection.

Embodiment 103: The method of embodiment 101, comprising locally administering within the 24-hour period 10 or more doses of the at least one antibiotic at a concentration that exceeds the MBEC level for the at least one antibiotic for the local infection.

Embodiment 104: The method of embodiment 101, comprising locally administering within the 24-hour period 20 or more doses of at least one antibiotic at a concentration that exceeds the MBEC level for the at least one antibiotic for the local infection.

Embodiment 105: The method of embodiment 101, wherein the at least one antibiotic comprises two or more antibiotics.

Embodiment 106: The method of embodiment 105, wherein the two or more antibiotics are selected from the group consisting of vancomycin, daptomycin, and tobramycin.

Embodiment 107: The method of embodiment 105, wherein there is at least a 3-log, 4-log, 5-log, or 6-log reduction in the number of CFU's of the local infection within 7 days.

Embodiment 108: The method of embodiment 105, wherein greater than 99% of CFU's of the local infection are killed within 7 days.

Embodiment 109: The method of embodiment 105, wherein greater than 99% of CFU's of the local infection are killed within 7 days.

Embodiment 110: The method of embodiment 105, wherein there is at least a 3-log, 4-log, 5-log, or 6-log reduction in the number of CFU's of the local infection within 3 days.

Embodiment 111: The method of embodiment 105, wherein greater than 99% of CFU's of the local infection are killed within 3 days.

Embodiment 112: The method of embodiment 105, wherein greater than 99% of CFU's of the local infection are killed within 3 days.

Embodiment 113: The method of embodiment 98, wherein there is at least a 3-log, 4-log, 5-log, or 6-log reduction in the number of CFU's of the local infection within 24 hours.

Embodiment 114: The method of embodiment 105, wherein greater than 99% of CFU's of the local infection are killed within 24 days.

Embodiment 115: The method of embodiment 105, wherein greater than 99% of CFU's of the local infection are killed within 24 days.

Embodiment 116: A method of treating a localized infection of a human patient, the method comprising: locally administering to the human patient two or more doses of at least one antibiotic within 24 hours, wherein an AUC/MBEC is maintained at the site of the localized infection for at least 5 hours a day.

Embodiment 117: The method of embodiment 116, wherein the AUC/MBEC is maintained for at least 10 hours a day.

Embodiment 118: The method of embodiment 116, wherein the AUC/MBEC is maintained for at least 20 hours a day.

Embodiment 119: A method of treating a localized infection in a human patient, the method comprising: locally administering to the human patient for a period of two or more days, at least one antibiotic at a concentration that exceeds a MBEC for the at least one antibiotic for the local infection for at least a first day, and the at least one antibiotic at a concentration less than the concentration of the at least one antibiotic on the first day, wherein the concentration of the at least one antibiotic on subsequent days exceeds a minimum inhibitory concentration of the at least one antibiotic for the local infection.

Embodiment 120: The method of embodiment 119, wherein the period of two or more days is a period of 5-7 days and the at least first day is 1-3 days.

Embodiment 121: A method of treating a local infection of a human patient in need thereof, the method comprising: administering by local irrigation to the human patient at least a 25 mg dose of vancomycin at a concentration of 3,000 mcg/mL or greater; and providing less than a 30 mcg/mL trough serum concentration in the patient for 24 hours following administration.

Embodiment 122: A method of treating a local infection of a human patient in need thereof, the method comprising: administering by local irrigation to the human patient a dosage of vancomycin and tobramycin, wherein the dosage comprises: at least a 50 mg dose of vancomycin at a concentration of 3,000 mcg/mL or greater; and at least a 50 mg dose of tobramycin at a concentration of 2,000 mcg/mL or greater, and wherein the administering provides a trough serum concentration less than 30 mcg/mL of vancomycin and a peak serum concentration less than 10 mcg/mL of tobramycin in the patient for 24 hours following administration.

Embodiment 123: A method of treating a localized infection in a human patient, the method comprising: identifying at least one microbe within the localized infection; and locally administering to the human patient two or more antibiotics, wherein at least one of the antibiotics is administered at a concentration that exceeds a MBEC level for the at least one antibiotic for the at least one microbe.

Embodiment 124: The method of embodiment 123, wherein when the microbe is one or more of Staphylococcus aureus, Staphylococcus epidermidis, Enterococcus, Escherichia coli, and Pseudomonas aeruginosa, the two or more antibiotics are vancomycin and tobramycin.

Embodiment 125: The method of embodiment 123, wherein when the microbe is methicillin resistant Staphylococcus aureus (MRSA) or vancomycin resistant Enterococcus faecalis (VRE), the two or more antibiotics are daptomycin and tobramycin.

Embodiment 126: The method of embodiment 123, wherein the at least one microbe is identified by laboratory testing of a sample from the site of the localized infection.

Embodiment 127: The method of embodiment 123, wherein there is at least a 4-log reduction in the number of CFU's of the local infection within 7 days of administering.

Embodiment 128: The method of embodiment 127, wherein there is a ≥5-log, ≥6-log, or ≥7-log reduction in the number of CFU's of the local infection within 7 days of administering.

Embodiment 129: The method of embodiment 123, wherein greater than 99% of CFU's of the local infection are killed within 7 days of administering.

Embodiment 130: The method of embodiment 129, wherein greater than 99.9% of CFU's of the local infection are killed within 7 days of administering.

Embodiment 131: The method of embodiment 123, wherein there is a 4-log reduction in the number of CFU's of the local infection within 24 hours of administering.

Embodiment 132: The method of embodiment 131, wherein there is a ≥5-log, ≥6-log, or ≥7-log reduction in the number of CFU's of the local infection within 24 hours of administering.

Embodiment 133: The method of embodiment 123, wherein greater than 99% of CFU's of the local infection are killed within 24 hours of administering.

Embodiment 134: The method of embodiment 133, wherein greater than 99.9% of CFU's of the local infection are killed within 24 hours of administering.

Embodiment 135: A method of treating a localized infection in a human patient, the method comprising: locally administering to the human patient two or more antibiotics, wherein at least one of the antibiotics is administered at a concentration that exceeds a MBEC level for the at least one antibiotic for the at least one microbe; identifying, during the administering, at least one microbe within the localized infection; and switching at least one of the antibiotics to a different antibiotic after identifying the at least one microbe.

Embodiment 136: The method of embodiment 135, wherein the at least one microbe is identified by laboratory testing of a sample from the site of the localized infection.

Embodiment 137: The method of embodiment 135, wherein there is at least a 4-log reduction in the number of CFU's of the local infection within 7 days of administering.

Embodiment 138: The method of embodiment 135, wherein there is a ≥5-log, ≥6-log, or ≥7-log reduction in the number of CFU's of the local infection within 7 days of administering.

Embodiment 139: The method of embodiment 135, wherein greater than 99% of CFU's of the local infection are killed within 7 days of administering.

Embodiment 140: The method of embodiment 139, wherein greater than 99.9% of CFU's of the local infection are killed within 7 days of administering.

Embodiment 141: The method of embodiment 135, wherein there is a ≥4-log reduction in the number of CFU's of the local infection within 24 hours of administering.

Embodiment 142: The method of embodiment 141, wherein there is a ≥5-log, ≥6-log, or ≥7-log reduction in the number of CFU's of the local infection within 24 hours of administering.

Embodiment 143: The method of embodiment 135, wherein greater than 99% of CFU's of the local infection are killed within 24 hours of administering.

Embodiment 144: The method of embodiment 143, wherein greater than 99.9% of CFU's of the local infection are killed within 24 hours of administering.

Embodiment 145: A method of treating a localized infection in a human patient, the method comprising: locally administering to the human patient two or more antibiotics, wherein at least one of the antibiotics is administered at a concentration that exceeds a MBEC level for the at least one antibiotic for the at least one microbe; identifying, during the administering, at least one microbe within the localized infection; and switching at least one of the antibiotics to a different antibiotic after identifying the at least one microbe.

Embodiment 146: The method of embodiment 145, wherein the second antibiotic combination results in an improvement of ≥0.5-log, ≥1-log, ≥1.5-log, ≥2.0-log, ≥2.5-log, ≥ 3.0-log, ≥3.5-log, ≥ or 4.0-log of the identified at least one microbe as compared to the first antibiotic combination against the identified microbe.

Embodiment 147: The method of embodiment 145, wherein the at least one microbe is identified by laboratory testing of a sample from the site of the localized infection.

Embodiment 148: The method of embodiment 145, wherein there is at least a 4-log reduction in the number of CFU's of the local infection within 7 days of administering the second antibiotic combination.

Embodiment 149: The method of embodiment 148, wherein there is a ≥5-log, ≥6-log, or ≥7-log reduction in the number of CFU's of the local infection within 7 days of administering the second antibiotic combination.

Embodiment 150: The method of embodiment 145, wherein greater than 99% of CFU's of the local infection are killed within 7 days of administering the second antibiotic combination.

Embodiment 151: The method of embodiment 145, wherein greater than 99.9% of CFU's of the local infection are killed within 7 days of administering the second antibiotic combination.

Embodiment 152: The method of embodiment 145, wherein there is a ≥4-log reduction in the number of CFU's of the local infection within 24 hours of administering the second antibiotic combination.

Embodiment 153: The method of embodiment 152, wherein there is a ≥5-log, ≥6-log, or ≥7-log reduction in the number of CFU's of the local infection within 24 hours of administering the second antibiotic combination.

Embodiment 154: The method of embodiment 145, wherein greater than 99% of CFU's of the local infection are killed within 24 hours of administering the second antibiotic combination.

Embodiment 155: The method of embodiment 145, wherein greater than 99.9% of CFU's of the local infection are killed within 24 hours of administering the second antibiotic combination.

1. A method of treating a localized infection of a human patient, the method comprising: locally administering a solution to the human patient in a 24-hour period a dose of at least one antimicrobial agent that substantially exceeds a maximum recommended daily systemic dose of the at least one antimicrobial agent; wherein the locally administering comprises irrigating the localized infection with the at least one antimicrobial agent one or more times and wherein during the 24-hour period the administering provides a peak or trough serum concentration less than or equal to a maximum safe level for serum concentration of the at least one antimicrobial agent.

2. (canceled)

3. The method of claim 1, wherein the irrigating the localized infection comprises soaking the local site of infection with the at least one antimicrobial agent and subsequently aspirating the at least one antimicrobial agent from the local site of infection.

4. The method of claim 3, wherein the soaking is for about 10 minutes to about 180 minutes.

5. (canceled)

6. The method of claim 1, wherein the irrigating is performed at least twice in the 24-hour period.

7-10. (canceled)

11. The method of claim 1, wherein the locally administered dose of the at least one antimicrobial agent in the 24-hour period exceeds the maximum recommended daily systemic dose of the at least one antimicrobial agent by at least 125%.

12-14. (canceled)

15. The method of claim 1, wherein the at least one antimicrobial agent is an antibiotic.

16. The method of claim 15, wherein the at least one antibiotic is an aminoglycoside or a glycopeptide.

17. (canceled)

18. The method of claim 16, wherein the at least one antibiotic is vancomycin.

19-22. (canceled)

23. The method of claim 18, wherein the locally administered daily dose of vancomycin is 3,000 to 30,000 mg in the 24-hour period, and the trough serum concentration of vancomycin is less than 30 mcg/mL.

24-26. (canceled)

27. The method of claim 18, wherein locally administering comprises irrigating the localized infection with the vancomycin two or more times in the 24-hour period.

28-39. (canceled)

40. The method of claim 16, wherein the at least one antibiotic is tobramycin.

41-44. (canceled)

45. The method of claim 40, wherein the daily dose of tobramycin is 100 to 1,000 mg/day, and the peak serum concentration of tobramycin is less than 10 mcg/mL.

46-49. (canceled)

50. The method of claim 1, wherein the at least one antimicrobial agent comprises two antibiotics.

51. The method of claim 50, wherein the two antibiotics comprise vancomycin and tobramycin.

52. The method of claim 51, wherein the two antibiotics are sequentially administered.

53. The method of claim 52, wherein the vancomycin is locally administered hourly for 20 to 22 hours in the 24-hour period, wherein the vancomycin soaks for about 30 minutes and subsequently aspirated for about 30 minutes, and wherein the tobramycin is locally administered once in the 24-hour period for about 2 hours.

54. The method of claim 51, wherein the locally administered concentration of the vancomycin is 3,000 mcg/mL to 30,000 mcg/mL and the locally administered concentration of the tobramycin is 2,000 mcg/mL to 20,000 mcg/mL.

55. The method of claim 51, wherein the local administration is provided for 1 to 10 days.

56. The method of claim 51, wherein the vancomycin and tobramycin are administered at a fixed dose ratio of 6:1 to 150:1.

57-59. (canceled)

60. The method of claim 51, wherein the daily dose of vancomycin is 3,000 to 30,000 mg in the 24-hour period and the daily dose of tobramycin is 100 to 1,000 mg in the 24-hour period, and the trough serum concentration of vancomycin is less than 30 mcg/mL and the peak serum concentration of tobramycin is less than 10 mcg/mL.

61-71. (canceled)

72. A method of treating a musculoskeletal infection of a human patient comprising: administering to the human patient at least one antimicrobial agent via irrigation at a locally administered concentration that exceeds a minimum biofilm eradication concentration (MBEC) of the at least one antimicrobial agent; wherein the administering provides a peak or trough serum concentration of the at least one antimicrobial agent less than a maximum safe serum concentration for the at least one antimicrobial agent.

73. The method of claim 72, wherein the MBEC comprises the concentration at which the at least one antimicrobial agent reduces the number of colony forming units (CFU's) of a specific microbe that has formed a biofilm for at least 24 hours by greater than 99.999% within a period of time over which the at least one antimicrobial agent is locally administered to the localized infection.

74. The method of claim 73, wherein the microbe is one or more of Staphylococcus aureus, methicillin resistant Staphylococcus aureus (MRSA), vancomycin resistant Enterococcus faecalis (VRE), Staphylococcus epidermidis, Enterococcus, Escherichia coli, and Pseudomonas aeruginosa.

75. The method of claim 72, wherein the at least one antimicrobial agent comprises a vancomycin and tobramycin.

76-77. (canceled)

78. The method of claim 75, wherein the locally administered concentration of vancomycin is 3,000 to 50,0000 mcg/mL and the locally administered concentration of tobramycin is 100 to 40,000 mcg/mL, and the peak serum concentration of tobramycin is less than 10 mcg/mL and the trough serum concentration of vancomycin is less than 30 mcg/mL.

79-83. (canceled)

84. A method of treating a localized infection in a human patient, the method comprising: locally administering to the human patient, in a 24-hour period, a total dose of vancomycin that exceeds 3,000 mg/day.

85. The method of claim 84, wherein the total dose of vancomycin in the 24-hour period is
a) at least 6,000 mg/day;
b) at least 12,000 mg/day; or
c) at least 24,000 mg/day.

86-87. (canceled)

88. A method of treating a localized infection in a human patient, the method comprising: locally administering to the human patient, in a 24-hour period, a total dose of tobramycin that exceeds 100 mg in the 24-hour period.

89. The method of claim 88, wherein the dose of tobramycin is
a) at least 200 mg in the 24-hour period;
b) at least 400 mg in the 24-hour period; or
c) at least 800 mg in the 24-hour period.

90-161. (canceled)
